(19) United States
(12) Patent Application Publication
(10) Pub. No.: US 2008/0070883 A1
(43) Pub. Date: Mar. 20, 2008

(54) Use of LXR modulators for the prevention and treatment of skin aging

(75) Inventor: Sunil Nagpal, Collegeville, PA (US)

Correspondence Address: POTTER ANDERSON & CORROON LLP/WYETH, 1313 NORTH MARKET STREET, HERCULES PLAZA, SIXTH FLOOR, WILMINGTON, DE, 19801, US

(73) Assignee: Wyeth, Madison, NJ (US)

(21) Appl. No.: 11/901,514

(22) Filed: Sep. 18, 2007

Related U.S. Application Data
(60) Provisional application No. 60/845,685, filed on Sep. 19, 2006.

(51) Int. Cl. A61K 31/56; A61Q 19/08

(52) U.S. Cl. 514/182

(57) ABSTRACT

Disclosed herein are compounds for preventing or treating skin aging through the use of LXR modulators and methods of use thereof.

### CROSS-REFERENCE TO RELATED APPLICATION

This application claims priority from U.S. Provisional Application No. 60/845,685 filed Sep. 19, 2006, which is incorporated by reference herein in its entirety.

### FIELD OF THE INVENTION

The present invention relates to compounds for treating or preventing skin aging with LXR modulators and methods of use thereof.

### BACKGROUND OF THE INVENTION

Genetically programmed chronological aging is a complex process at the molecular level and is influenced by telomere shortening and damage to cellular DNA. Structurally, dermal tissues become more rigid and the skin's elastic fibers fragment and collagen depletes. Elastogenic polypeptides in the elastic fibers of the connective tissue degenerate over time, and this degeneration essentially causes aging of the skin as the elastic fibers that were once in situ maintained elasticity and skin tone. Recently, skin aging has been hypothesized to result from the association of seven factors: 1) chronological factors, 2) genetic factors, 3) exposure to ultraviolet rays (photodamage), 4)-behavioral factors, 5) endocrinological factors, 6) catabolic factors, and 7) mechanical factors (Jellouli Elloumi A et al., Tunis. Med. 79:1-9 (2001)). The hidden forces of gravity also pull on skin tissue contributing to the symptoms of aging. The more recognized causes of skin aging are generally concerned with photodamage and pollution.

Currently available approaches to treating or preventing wrinkles are either injectable (e.g., Botox®) or have teratogenicity and skin irritation, flaking, and redness side effects (e.g., retinoids). Botox® (Botulinum toxin Type A) is a bacterial toxin used primarily as a muscle relaxant, but it is the only serotype A botulinum (Allergan, Irvine, Calif.) available for clinical use in select territories for the treatment of facial lines, crows feet, and wrinkles. Dermatologists use purified botulinum toxin in very small amounts to inject into a targeted immobilization of muscle movement, which prevents lines from forming when the patient frowns or squints. Retin-A® (tretinoin), a retinoid, is more commonly used as a treatment for acne. In this indication, Retin-A® reduces the formation of acne spots and promotes the rapid healing of visible acne. Retin-A® also has an off-label use in skin aging. Renova®/Retinova (tretinoin) is indicated for fine facial lines and wrinkles as part of a comprehensive skin care program. Restylane® (hyaluronic acid filler injections) has been used in more than three million treatments in over 70 countries and was approved in the U.S. in December 2003 for the treatment of facial wrinkles and folds. Other hyaluronic acid fillers include Hylaform® and Captique®.

Liver X receptors (LXRs), originally identified from liver as orphan receptors, are members of the nuclear hormone receptor super family and are expressed in skin, for example in keratinocytes, and granulocytes. LXRs are ligand-activated transcription factors and bind to DNA as obligate heterodimers with retinoid X receptors (RXRs). LXRs activated by oxysterols (endogenous ligands) display potent anti-inflammatory properties in vitro and in vivo. Topical application of LXR ligands inhibits inflammation in murine models of contact (oxazolone-induced) and irritant (TPA-induced) dermatitis.

### SUMMARY OF THE INVENTION

One aspect is for an anti-skin aging composition comprising a therapeutically effective amount of an LXR modulator.

Another aspect is for a method for the treatment of skin aging comprising administering to a mammal in need thereof a therapeutically effective amount of an LXR modulator.

A further aspect relates to a method for the prevention of skin aging comprising administering to a mammal a therapeutically effective amount of an LXR modulator.

An additional aspect is for a method of counteracting UV photodamage comprising contacting a skin cell exposed to UV light with a therapeutically effective amount of an LXR modulator.

Another aspect relates to a method of identifying an LXR modulator capable of inducing an anti-skin aging effect comprising: (a) providing a sample containing LXR; (b) contacting the sample with a test compound; and (c) determining whether the test compound induces TIMP1 expression, induces ASAH1 expression, induces SPTLC1 expression, induces SMPD1 expression, induces LASS2 expression, induces TXNRD1 expression, induces GPX3 expression, induces GSR expression, induces CAT expression, induces ABCA1 expression, induces ABCA2 expression, induces ABCA12 expression, induces ABCA13 expression, induces ABCG1 expression, induces decorin expression, inhibits TNFα expression, inhibits MMP1 expression, inhibits MMP3 expression, inhibits IL-8 expression, or a combination thereof.

Other aspects and advantages of the present invention will become apparent to those skilled in the art upon reference to the detailed description that hereinafter follows.

### BRIEF DESCRIPTION OF THE FIGURES

FIG. 1A is a bar graph illustrating that UV inhibits, and LXR modulator induces, LXRα expression in Normal Human Epidermal Keratinocytes (NHEKs). FIG. 1B is a bar graph illustrating that UV inhibits, and LXR modulator induces, LXRβ expression in NHEKs. V=vehicle; UV=ultraviolet light; T1317=Tularik 0901317.

FIG. 2 is a bar graph illustrating that UV-induced TNFα expression in NHEKs is inhibited by an LXR modulator. V=vehicle; UV=ultraviolet light; T1317=Tularik 0901317.

FIG. 3 is a bar graph illustrating that UV-induced MMP3 expression in NHEKs is inhibited by an LXR modulator. V=vehicle; UV=ultraviolet light; T1317=Tularik 0901317.

FIG. 4 is a bar graph illustrating that TIMP1 expression is up-regulated by an LXR modulator in NHEKs. V=vehicle; UV=ultraviolet light; T1317=Tularik 0901317.

FIG. 5 is a bar graph illustrating that UV-induced IL-8 expression in NHEKs is down-regulated by an LXR modulator. V=vehicle; UV=ultraviolet light; T1317=Tularik 0901317.

FIG. 6A is a bar graph illustrating that an LXR modulator induces the expression of ABCA1, ABCA2, ABCA12, ABCA13, and ABCG1 in NHEKs.

FIG. 6B is a bar graph illustrating that an LXR modulator relieves UV-mediated inhibition of ABCA12 in NHEKs. V=vehicle; UV=ultraviolet light; T1317=Tularik 0901317.

FIG. 7 is a bar graph illustrating that an LXR modulator relieves UV-mediated inhibition of decorin in NHEKs. V=vehicle; UV=ultraviolet light; T1317=Tularik 0901317.

FIG. 8A is a bar graph illustrating that an LXR modulator inhibits MMP1 in fibroblasts.

FIG. 8B is a bar graph illustrating that an LXR modulator inhibits MMP3 in fibroblasts. V=vehicle; T1317=Tularik 0901317.

FIG. 9 is a bar graph illustrating that an LXR modulator induces the expression of TIMP1 in fibroblasts. V=vehicle; T1317=Tularik 0901317.

FIG. 10A is a bar graph illustrating that an LXR modulator induces expression of acid ceramidase (ASAH1), serine palmitoyl transferase (SPTLC1), sphingomyelin phosphodiesterase (SMPD1), and ceramide synthase (LASS2) in keratinocytes (NHEKs). T1317=Tularik 0901317.

FIG. 10B illustrates the sphingosine synthesis pathway.

FIG. 11 is a bar graph illustrating that an LXR modulator induces expression of thioredoxin reductase (TXNRD1), glutathione peroxidase (GPX3), glutathione reductase (GSR), and catalase (CAT) in keratinocytes (NHEKs). T1317=Tularik 0901317.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Here, Applicants disclose the topical use of an LXR modulator for the prevention and treatment of skin aging. Applicants demonstrate herein that LXR modulators inhibit the expression of metalloproteases that degrade skin collagen and elastin. In addition, LXR modulators are expected to induce the expression of type I collagen. Increased keratinocyte lipogenesis and differentiation by the LXR modulator in skin will also help in improvement in barrier formation.

Applicants also demonstrate herein that LXR expression is up-regulated by the LXR modulator in UV-induced keratinocytes. An LXR modulator inhibits UV-induced TNFα expression in immortalized keratinocytes. LXR modulator also inhibits MMP1 and MMP3 expression in TNFα activated keratinocytes. Further, LXR modulator induces the expression of TIMP1 in keratinocytes and fibroblasts. Therefore, LXR appears to be a novel target for the treatment of skin aging. On the other hand, LXR ligands do not inhibit AP1-dependent gene expression. Therefore, LXR modulators may not inhibit keratinocyte differentiation and cause skin thinning.

Treatment or prevention of skin aging using LXR modulator should be more efficacious and easier to administer compared to current injectable methods, and should be devoid of the classical retinoid side-effects.

I. Definitions

In the context of this disclosure, a number of terms shall be utilized.

As used herein, the term “about” or “approximately” means within 20%, preferably within 10%, and more preferably within 5% of a given value or range.

The term a “therapeutically effective amount” as used herein refers to the amount of an LXR modulator that, when administered to a mammal in need, is effective to at least partially ameliorate or to at least partially prevent conditions related to skin aging.

As used herein, the term “expression” includes the process by which polynucleotides are transcribed into mRNA and translated into peptides, polypeptides, or proteins.

The terms “induce” or “induction” of TIMP1, ASAH1, SPTLC1, SMPD1, LASS2, TXNRD1, GPX3, GSR, CAT, ABCA1, ABCA2, ABCA12, ABCA13, ABCG1, or decorin expression refer to an increase, induction, or otherwise augmentation of TIMP1, ASAH1, SPTLC1, SMPD1, LASS2, TXNRD1, GPX3, GSR, CAT, ABCA1, ABCA2, ABCA12, ABCA13, ABCG1, or decorin mRNA and/or protein expression. The increase, induction, or augmentation can be measured by one of the assays provided herein. Induction of TIMP1, ASAH1, SPTLC1, SMPD1, LASS2, TXNRD1, GPX3, GSR, CAT, ABCA1, ABCA2, ABCA12, ABCA13, ABCG1, or decorin expression does not necessarily indicate maximal expression of TIMP1, ASAH1, SPTLC1, SMPD1, LASS2, TXNRD1, GPX3, GSR, CAT, ABCA1, ABCA2, ABCA12, ABCA13, ABCG1, or decorin. An increase in TIMP1, ABCA12, or decorin expression can be, for example, at least about 10%, 20%, 30%, 40%, 50%, 60%, 70%, 80%, 90% or more. In one embodiment, induction is measured by comparing TIMP1, ASAH1, SPTLC1, SMPD1, LASS2, TXNRD1, GPX3, GSR, CAT, ABCA1, ABCA2, ABCA12, ABCA13, ABCG1, or decorin mRNA expression levels from untreated keratinocytes to that of TIMP1, ASAH1, SPTLC1, SMPD1, LASS2, TXNRD1, GPX3, GSR, CAT, ABCA1, ABCA2, ABCA12, ABCA13, ABCG1, or decorin mRNA expression levels from LXR modulator-treated keratinocytes.

The terms “inhibit” or “inhibition” of TNFα , MMP1, MMP3, or IL-8 expression refer to a reduction, inhibition, or otherwise diminution of TNFα, MMP1, MMP3, or IL-8 mRNA and/or protein expression. The reduction, inhibition, or diminution of binding can be measured by one of the assays provided herein. Inhibition of TNFα, MMP1, MMP3, or IL-8 expression does not necessarily indicate a complete negation of TNFα, MMP1, MMP3, or IL-8 expression. A reduction in expression can be, for example, at least about 10%, 20%, 30%, 40%, 50%, 60%, 70%, 80%, 90% or more. In one embodiment, inhibition is measured by comparing TNFα, MMP1, MMP3, or IL-8 mRNA expression levels from untreated keratinocytes to that of TNFα, MMP1, MMP3, or IL-8 mRNA expression levels from LXR modulator-treated keratinocytes.

“Liver X receptor” or “LXR” refers to both LXRα and LXRβ, and variants, isoforms, and active fragments thereof. LXRβ is ubiquitously expressed, while LXRα expression is limited to liver, kidney, intestine, spleen, adipose tissue, macrophages, skeletal muscle, and, as demonstrated herein, skin. Representative GenBank® accession numbers for LXRα sequences include the following: human (Homo sapiens, Q13133), mouse (Mus musculus, Q9Z0Y9), rat (Rattus norvegicus, Q62685), cow (Bos taurus, Q5E9B6), pig (Sus scrofa, AAY43056), chicken (Gallus gallus, AAM90897). Representative GenBank® accession numbers for LXRβ include the following: human (Homo sapiens, P55055), mouse (Mus musculus, Q60644), rat (Rattus norvegicus, Q62755), cow (Bos taurus, Q5BIS6).

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

The term “modulate” encompasses either a decrease or an increase in activity or expression depending on the target molecule. For example, a TIMP1 modulator is considered to modulate the expression of TIMP1 if the presence of such TIMP1 modulator results in an increase or decrease in TIMP1 expression.

“Proinflammatory cytokine” as used herein refers to any cytokine that can activate cytotoxic, inflammatory, or delayed hypersensitivity reactions. Exemplary proinflammatory cytokines include colony stimulating factors (CSFs), for example granulocyte-macrophage CSF, granulocyte CSF, erythropoietin; transforming growth factors (TGFs), for example TGFβ; interferons (IFNs), for example IFNα, IFNβ, IFNγ; interleukins (ILs), for example IL-1α, IL-1β, IL-3, IL-6, IL-7, IL-8, IL-9, IL-11, IL-12, IL-15; tumor necrosis factors (TNFs), for example TNFα, TNFβ; adherence proteins, for example intracellular adhesion molecule (ICAM), vascular cell adhesion molecule (VCAM); growth factors, for example leukemia inhibitory factor (LIF), macrophage migration-inhibiting factor (MIF), epidermal growth factor (EGF), platelet-derived growth factor (PDGF), fibroblast growth factor (FGF), insulin-like growth factor (IGF), nerve growth factor (NGF), B-cell growth factor (BCGF); chemokines, for example monocyte chemoattractant proteins (MCP-1, MCP-2, MCP-3), macrophage inflammatory protein (MIP), growth-related oncogene, gamma interferon-inducible protein; leukotrienes, for example leukotriene B₄, leukotrine D₄; vasoactive factors, for example histamine, bradykinin, platelet activating factor (PAF); prostaglandins, for example prostaglandin E₂.

The term “skin aging” includes conditions derived from intrinsic chronological aging (for example, deepened expression lines, reduction of skin thickness, inelasticity, and/or unblemished smooth surface), those derived from photoaging (for example, deep wrinkles, yellow and leathery surface, hardening of the skin, elastosis, roughness, dyspigmentations (age spots) and/or blotchy skin), and those derived from steroid-induced skin thinning.

II. LXR Modulators

Preferred compounds will be LXR modulators with LXRα and/or LXRβ modulator activities. The term “LXR modulator” includes LXRα and/or LXRβ agonists, antagonists and tissue selective LXR modulators, as well as other agents that induce the expression and/or protein levels of LXRs in the skin cells.

LXR modulators useful in the present invention include natural oxysterols, synthetic oxysterols, synthetic nonoxysterols, and natural nonoxysterols. Exemplary natural oxysterols include 20(S) hydroxycholesterol, 22(R) hydroxycholesterol, 24(S) hydroxycholesterol, 25-hydroxycholesterol, 24(S), 25 epoxycholesterol, and 27-hydroxycholesterol. Exemplary synthetic oxysterols include N,N-dimethyl-3β-hydroxycholenamide (DMHCA). Exemplary synthetic nonoxysterols include N-(2,2,2-trifluoroethyl)-N-{4-[2,2,2-trifluoro-1-hydroxy-1-(trifluoromethyl)ethyl]phenyl}benzene sulfonamide (TO901317; Tularik 0901317), [3-(3-(2-chloro-trifluoromethylbenzyl-2,2-diphenylethylamino)propoxy)phenylacetic acid] (GW3965), N-methyl-N-[4-(2,2,2-trifluoro-1-hydroxy-1-trifluoromethyl-1-ethyl)-phenyl]-benzenesulfonamide (TO314407), 4,5-dihydro-1-(3-(3-trifluoromethyl-7-propyl-benzisoxazol-6-yloxy)propyl)-2,6-pyrimidinedione, 3-chloro-4-(3-(7-propyl-3-trifluoromethyl-6-(4,5)-isoxazolyl)propylthio)-phenyl acetic acid (F₃MethylAA), and acetyl-podocarpic dimer. Exemplary natural nonoxysterols include paxilline, desmosterol, and stigmasterol.

Other useful LXR modulators are disclosed, for example, in Published U.S. Patent Application Nos. 2005/0036992, 2005/0080111, 2003/0181420, 2003/0086923, 2003/0207898, 2004/0110947, 2004/0087632, 2005/0009837, 2004/0048920, and 2005/0123580; U.S. Pat. Nos. 6,316,503, 6,828,446, 6,822,120, and 6,900,244; WO01/41704; Menke J G et al., Endocrinology 143:2548-58 (2002); Joseph S B et al., Proc. Natl. Acad. Sci. USA 99:7604-09 (2002); Fu X et al., J. Biol. Chem. 276:38378-87 (2001); Schultz J R et al., Genes Dev. 14:2831-38 (2000); Sparrow C P et al., J. Biol. Chem. 277:10021-27 (2002); Yang C et al., J. Biol. Chem., Manuscript M603781200 (Jul. 20, 2006); Bramlett K S et al., J. Pharmacol. Exp. Ther. 307:291-96 (2003); Ondeyka J G et al., J. Antibiot (Tokyo) 58:559-65 (2005).

Additionally, compounds disclosed in co-owned, copending U.S. patent application Ser. No. 11/365,750 are useful in the therapeutic or pharmaceutical compositions disclosed herein. Compounds disclosed therein include those having formula (I): 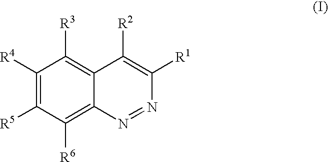
in which:
R¹ can be:
(i) hydrogen; or
(ii) C₁-C₂₀ alkyl or C₁-C₂₀ haloalkyl, each of which is optionally substituted with from 1-10 R^{a}; or
(iii) C₆-C₁₈ aryl or heteroaryl including 5-16 atoms, each of which is optionally substituted with from 1-10 R^{b}; or
(iv) C₇-C₂₀ aralkyl or heteroaralkyl including 6-20 atoms, each of which is optionally substituted with from 1-10 R^{c}; or
(v) C₂-C₂₀ alkenyl or C₂-C₂₀ alkynyl, each of which is optionally substituted with from 1-10 R^{d};
(vi) C₃-C₂₀ cycloalkyl or C₃-C₂₀ halocycloalkyl, optionally substituted with from 1-10 R^{e}; or
(vii) C₃-C₂₀ cycloalkenyl, heterocyclyl including 3-20 atoms, or heterocycloalkenyl including 3-20 atoms, each of which is optionally substituted with from 1-10 R^{f}; or
(viii) —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h});
   R² can be:
(i) C₆-C₁₈ aryl or heteroaryl including 5-16 atoms, each of which is optionally substituted with from 1-10 R^{b}; or
(ii) C₇-C₂₀ aralkyl or heteroaralkyl including 6-20 atoms, each of which is optionally substituted with from 1-10 R^{c}; or
(iii) C₃-C₂₀ cycloalkyl or C₃-C₂₀ halocycloalkyl, optionally substituted with from 1-10 R^{e}; or
(iv) C₃-C₂₀ cycloalkenyl, heterocyclyl including 3-20 atoms, or heterocycloalkenyl including 3-20 atoms, each of which is optionally substituted with from 1-10 R^{f};
   each of R³, R⁴, R⁵, and R⁶ can be, independently:
(i) hydrogen, halo; NR^{g}R^{h}; nitro; azido, hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{b}; C₇-C₂₀ aralkoxy or heteroaralkoxy including 6-20 atoms, each of which is optionally substituted with from 1-10 R^{c}; C₃-C₂₀ cycloalkoxy or C₃-C₂₀ halocycloalkoxy, each of which is optionally substituted with from 1-10 R^{e}; C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{a}; C₆-C₁₈ thioaryloxy or thioheteroaryloxy including 5-16 atoms, each of which is optionally substituted with from 1-10 R^{b}; C₇-C₂₀ thioaralkoxy or thioheteroaralkoxy including 6-20 atoms, each of which is optionally substituted with from 1-10 R^{c}; C₃-C₂₀ thiocycloalkoxy or C₃-C₂₀ thiohalocycloalkoxy, each of which is optionally substituted with from 1-10 R^{e}; C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; formyl; C₁-C₃ alkylenedioxy; —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}); or
(ii) C₁-C₂₀ alkyl or C₁-C₂₀ haloalkyl, each of which is optionally substituted with from 1-10 R^{a}; or
(iii) C₃-C₂₀ cycloalkyl or C₃-C₂₀ halocycloalkyl, optionally substituted with from 1-10 R^{e}; or
(iv) C₃-C₂₀ cycloalkenyl, heterocyclyl including 3-20 atoms, or heterocycloalkenyl including 3-20 atoms, each of which is optionally substituted with from 1-10 R^{f}; or
(v) C₂-C₂₀ alkenyl or C₂-C₂₀ alkynyl, each of which is optionally substituted with from 1-10 R^{d}; or
(vi) C₇-C₂₀ aralkyl or heteroaralkyl including 6-20 atoms, each of which is optionally substituted with from 1-10 R^{c}; or
(vii) C₆-C₁₈ aryl or heteroaryl including 5-16 atoms, each of which is optionally substituted with from 1-10 R^{b};
R^{a} at each occurrence can be, independently NR^{g}R^{h}; nitro; azido; hydroxy; oxo; thioxo; ═NR^{m}; C₁-C₂₀ alkoxy; C₁-C₂₀ haloalkoxy; C₆-C₁₈ aryloxy or heteroaryloxy including 5-16 atoms, each of which is optionally substituted with from 1-10 R^{b}; C₇-C₂₀ aralkoxy or heteroaralkoxy including 6-20 atoms, each of which is optionally substituted with from 1-10 R^{c}; C₃-C₁₆ cycloalkoxy; C₃-C₁₆ halocycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{b}; C₇-C₂₀ thioaralkoxy or thioheteroaralkoxy including 6-20 atoms, each of which is optionally substituted with from 1-10 R^{c}; C₃-C₁₆ thiocycloalkoxy; C₃-C₁₆ thiohalocycloalkoxy; C₃-C₂₀ thiocycloalkenyloxy; thioheterocyclyloxy including 3-20 atoms; thioheterocycloalkenyloxy including 3-20 atoms; cyano; formyl; C₁-C₃ alkylenedioxy; —C(O)N R^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h});
   R^{b} at each occurrence can be, independently:
(i) halo; NR^{g}R^{h}; nitro; azido; hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{b} or R^{b′}; C₇-C₂₀ aralkoxy or heteroaralkoxy including 6-20 atoms, each of which is optionally substituted with from 1-10 R^{c}; C₃-C₁₆ cycloalkoxy or C₃-C₁₆ halocycloalkoxy, each of which is optionally substituted with from 1-10 R^{e}; C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{a}; C₆-C₁₈ thioaryloxy or thioheteroaryloxy including 5-16 atoms, each of which is optionally substituted with from 1-10 R^{b}; C₇-C₂₀ thioaralkoxy or thioheteroaralkoxy including 6-20 atoms, each of which is optionally substituted with from 1-10 R^{c}; C₃-C₁₆ thiocycloalkoxy or C₃-C₁₆ thiohalocycloalkoxy, each of which is optionally substituted with from 1-10 R^{e}; C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; formyl; C₁-C₃ alkylenedioxy; —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}); or
(ii) C₁-C₂₀ alkyl or C₁-C₂₀ haloalkyl, each of which is optionally substituted with from 1-10 R^{a}; or
(iii) C₃-C₂₀ cycloalkyl or C₃-C₂₀ halocycloalkyl, optionally substituted with from 1-10 R^{e}; or
(iv) C₃-C₂₀ cycloalkenyl, heterocyclyl including 3-20 atoms, or heterocycloalkenyl including 3-20 atoms, each of which is optionally substituted with from 1-10 R^{f}; or
(v) C₂-C₂₀ alkenyl or C₂-C₂₀ alkynyl, each of which is optionally substituted with from 1-10 R^{d}; or
(vi) C₇-C₂₀ aralkyl or heteroaralkyl including 6-20 atoms, each of which is optionally substituted with from 1-10 R^{c}; or
(vii) C₆-C₁₈ aryl or heteroaryl including 5-16 atoms, each of which is optionally substituted with from 1-10 R^{b′};
   R^{b′} at each occurrence can be, independently, halo; NR^{g}R^{h}; nitro; azido; hydroxy; C₁-C₂₀ alkyl, C₁-C₂₀ haloalkyl, C₂-C₂₀ alkenyl; C₂-C₂₀ alkynyl; C₃-C₂₀ cycloalkyl; C₃-C₂₀ halocycloalkyl; C₃-C₂₀ cycloalkenyl, heterocyclyl including 3-20 atoms; heterocycloalkenyl including 3-20 atoms; C₇-C₂₀ aralkyl; heteroaralkyl including 6-20 atoms; C₁-C₂₀ alkoxy; C₁-C₂₀ haloalkoxy; C₆-C₁₈ aryloxy or heteroaryloxy including 5-16 atoms; C₇-C₂₀ aralkoxy or heteroaralkoxy including 6-20 atoms; C₃-C₁₆ cycloalkoxy or C₃-C₁₆ halocycloalkoxy; C₃-C₂₀ cycloalkenyloxy, heterocyclyloxy including 3-20 atoms, or heterocycloalkenyloxy including 3-20 atoms; mercapto; C₁-C₂₀ thioalkoxy or C₁-C₂₀ thiohaloalkoxy; C₆-C₁₈ thioaryloxy or thioheteroaryloxy including 5-16 atoms; C₇-C₂₀ thioaralkoxy or thioheteroaralkoxy including 6-20 atoms; C₃-C₁₆ thiocycloalkoxy or C₃-C₁₆ thiohalocycloalkoxy; C₃-C₂₀ thiocycloalkenyloxy, thioheterocyclyloxy including 3-20 atoms, or thioheterocycloalkenyloxy including 3-20 atoms; cyano; formyl; C₁-C₃ alkylenedioxy; —C(O)N R^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h});
   R^{c} at each occurrence can be, independently:
(i) halo; NR^{g}R^{h}; nitro; azido; hydroxy; oxo; thioxo; ═NR^{m}; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{b}; C₇-C₂₀ aralkoxy or heteroaralkoxy including 6-20 atoms, each of which is optionally substituted with from 1-10 R^{c} or R^{c′}; C₃-C₁₆ cycloalkoxy or C₃-C₁₆ halocycloalkoxy, each of which is optionally substituted with from 1-10 R^{e}; C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{a}; C₆-C₁₈ thioaryloxy or thioheteroaryloxy including 5-16 atoms, each of which is optionally substituted with from 1-10 R^{b}; C₇-C₂₀ thioaralkoxy or thioheteroaralkoxy including 6-20 atoms, each of which is optionally substituted with from 1-10 R^{c}; C₃-C₁₆ thiocycloalkoxy or C₃-C₁₆ thiohalocycloalkoxy, each of which is optionally substituted with from 1-10 R^{e}; C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; formyl; C₁-C₃ alkylenedioxy; —C(O)N R^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}); or
(ii) C₁-C₂₀ alkyl or C₁-C₂₀ haloalkyl, each of which is optionally substituted with from 1-10 R^{a}; or
(iii) C₃-C₂₀ cycloalkyl or C₃-C₂₀ halocycloalkyl, optionally substituted with from 1-10 R^{e}; or
(iv) C₃-C₂₀ cycloalkenyl, heterocyclyl including 3-20 atoms, or heterocycloalkenyl including 3-20 atoms, each of which is optionally substituted with from 1-10 R^{f}; or
(v) C₂-C₂₀ alkenyl or C₂-C₂₀ alkynyl, each of which is optionally substituted with from 1-10 R^{d}; or
(vi) C₇-C₂₀ aralkyl or heteroaralkyl including 6-20 atoms, each of which is optionally substituted with from 1-10 R^{c} or R^{c′}; or
(vii) C₆-C₁₈ aryl or heteroaryl including 5-16 atoms, each of which is optionally substituted with from 1-10 R^{b};
R^{d} at each occurrence can be, independently, halo, NR^{g}R^{h}; nitro; azido; hydroxy; oxo; thioxo; ═NR^{m}; C₁-C₂₀ alkoxy; C₁-C₂₀ haloalkoxy; C₆-C₁₈ aryloxy; heteroaryloxy including 5-16 atoms; C₇-C₂₀ aralkoxy; heteroaralkoxy including 6-20 atoms; C₃-C₁₆ cycloalkoxy; C₃-C₁₆ halocycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₇-C₂₀ thioaralkoxy; thioheteroaralkoxy including 6-20 atoms; C₃-C₁₆ thiocycloalkoxy; C₃-C₁₆ thiohalocycloalkoxy; C₃-C₂₀ thiocycloalkenyloxy; thioheterocyclyloxy including 3-20 atoms; thioheterocycloalkenyloxy including 3-20 atoms; cyano; formyl; C₁-C₃ alkylenedioxy; —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h});
R^{c′} can be oxo; thioxo; ═NR^{m}; or R^{b′};
   R^{e} at each occurrence can be, independently:
(i) NR^{g}R^{h}; nitro; azido; hydroxy; oxo; thioxo; ═NR^{m}; C₁-C₂₀ alkoxy; C₁-C₂₀ haloalkoxy; C₆-C₁₈ aryloxy; heteroaryloxy including 5-16 atoms; C₇-C₂₀ aralkoxy; heteroaralkoxy including 6-20 atoms; C₃-C₁₆ cycloalkoxy; C₃-C₁₆ halocycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₇-C₂₀ thioaralkoxy; thioheteroaralkoxy including 6-20 atoms; C₃-C₁₆ thiocycloalkoxy; C₃-C₁₆ thiohalocycloalkoxy; C₃-C₂₀ thiocycloalkenyloxy; thioheterocyclyloxy including 3-20 atoms; thioheterocycloalkenyloxy including 3-20 atoms; cyano; formyl; C₁-C₃ alkylenedioxy; —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}); or
(ii) C₂-C₂₀ alkenyl or C₂-C₂₀ alkynyl, each of which is optionally substituted with from 1-10 R^{d}; or
(iii) C₆-C₁₈ aryl or heteroaryl including 5-16 atoms, each of which is optionally substituted with from 1-10 R^{b};
   R^{f} at each occurrence can be, independently:
(i) halo, NR^{g}R^{h}; nitro; azido; hydroxy; oxo; thioxo; ═NR^{m}; C₁-C₂₀ alkoxy; C₁-C₂₀ haloalkoxy; C₆-C₁₈ aryloxy; heteroaryloxy including 5-16 atoms; C₇-C₂₀ aralkoxy; heteroaralkoxy including 6-20 atoms; C₃-C₁₆ cycloalkoxy; C₃-C₁₆ halocycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₇-C₂₀ thioaralkoxy; thioheteroaralkoxy including 6-20 atoms; C₃-C₁₆ thiocycloalkoxy; C₃-C₁₆ thiohalocycloalkoxy; C₃-C₂₀ thiocycloalkenyloxy; thioheterocyclyloxy including 3-20 atoms; thioheterocycloalkenyloxy including 3-20 atoms; cyano; formyl; C₁-C₃ alkylenedioxy; —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}); or
(ii) C₂-C₂₀ alkenyl or C₂-C₂₀ alkynyl, each of which is optionally substituted with from 1-10 R^{a}; or
(iii) C₆-C₁₈ aryl or heteroaryl including 5-16 atoms, each of which is optionally substituted with from 1-10 R^{b};
   each of R^{g}, R^{h}, Rⁱ, and R^{j}, at each occurrence can be, independently:
(i) hydrogen; or
(ii) C₁-C₂₀ alkyl or C₁-C₂₀ haloalkyl, each of which is optionally substituted with from 1-10 R^{a};
(iii) C₂-C₂₀ alkenyl or C₂-C₂₀ alkynyl, each of which is optionally substituted with from 1-10 R^{d}; or
(iv) C₃-C₂₀ cycloalkyl or C₃-C₂₀ halocycloalkyl, each of which is optionally substituted with from 1-10 R^{e}; or
(v) C₃-C₂₀ cycloalkenyl, heterocyclyl including 3-16 atoms, or heterocycloalkenyl including 3-16 atoms, each of which is optionally substituted with from 1-10 R^{f}; or
(vi) C₇-C₂₀ aralkyl or heteroaralkyl including 6-20 atoms, each of which is optionally substituted with from 1-10 R^{c}; or
(vii) C₆-C₁₈ aryl or heteroaryl including 5-16 atoms, each of which is optionally substituted with from 1-10 R^{b}.
R^{k} can be Rⁱ, ORⁱ, or NR^{g}R^{h};
R^{m} can be hydrogen; C₁-C₁₂ alkyl or C₁-C₁₂ haloalkyl, each of which is optionally substituted with from 1-5 R^{a}; C₂-C₂₀ alkenyl; C₂-C₂₀ alkynyl; C₇-C₂₀ aralkyl; heteroaralkyl including 6-20 atoms; C₃-C₂₀ cycloalkyl; C₃-C₂₀ cycloalkenyl; heterocyclyl including 3-20 atoms; heterocycloalkenyl including 3-20 atoms; C₆-C₁₈ aryl; heteroaryl including 5-16 atoms; NR^{g}R^{h}, or ORⁱ; and n can be 0, 1 or 2; a compound of formula (I) can be a salt or a prodrug thereof (e.g., a pharmaceutically acceptable salt or prodrug thereof.

Also disclosed in U.S. patent application Ser. No. 11/365,750, and useful herein, are compounds having formula (II): 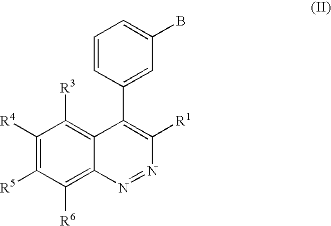
in which R¹, R³, R⁴, R⁵, and R⁶ can be as defined elsewhere, and B is:
(i) halo; NO₂; NR^{g}R^{h}; hydroxy; C₁-C₂₀ alkoxy optionally substituted with from 1-10 R^{a}; C₆-C₁₈ aryloxy or heteroaryloxy including 5-16 atoms, each of which is optionally substituted with from 1-10 R^{b′}; C₇-C₂₀ aralkoxy or heteroaralkoxy including 6-20 atoms, each of which is substituted with from 1-10 R^{c}; C₆-C₁₈ thioaryloxy or thioheteroaryloxy including 5-16 atoms, each of which is optionally substituted with from 1-10 R^{b′}; C₇-C₂₀ thioaralkoxy or thioheteroaralkoxy including 6-20 atoms, each of which is optionally substituted with from 1-10 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k}; or
(ii) C₁-C₂₀ alkyl or C₁-C₂₀ haloalkyl, each of which is optionally substituted with from 1-10 R^{a}; or
(iii) C₆-C₁₈ aryl or heteroaryl including 5-16 atoms, each of which is optionally substituted with from 1-10 R^{b′}.
(iv) C₇-C₂₀ aralkyl or heteroaralkyl including 6-20 atoms, each of which is optionally substituted with from 1-10 R^{c}; or
(v) hydrogen;
   in which R^{b′} and R^{c} can be as defined elsewhere; a compound of formula (V) can be a salt or prodrug thereof (e.g., a pharmaceutically acceptable salt or prodrug). Embodiments can include one more of the following features.
   R¹ can be:
(ii) C₁-C₂₀ alkyl or C₁-C₂₀ haloalkyl, each of which is optionally substituted with from 1-10 R^{a}; or
(iii) C₆-C₁₈ aryl or heteroaryl including 5-16 atoms, each of which is optionally substituted with from 1-10 R^{b}; or
(iv) C₇-C₂₀ aralkyl or heteroaralkyl including 6-20 atoms, each of which is optionally substituted with from 1-10 R^{c}; or
(viii) —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)OR; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}).
   R¹ can be:
(ii) C₁-C₁₆ alkyl or C₁-C₁₀ haloalkyl, each of which is optionally substituted with from 1-5 R^{a}; or
(iii) C₆-C₁₀ aryl or heteroaryl including 5-10 atoms, each of which is optionally substituted with from 1-5 R^{b}; or
(iv) C₇-C₁₆ aralkyl or heteroaralkyl including 6-16 atoms, each of which is optionally substituted with from 1-5 R^{c}; or
(viii) —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}).
   R¹ can be:
(ii) C₁-C₂₀ alkyl optionally substituted with from 1-10 R^{a}; or
(iii) C₆-C₁₈ aryl optionally substituted with from 1-10 R^{b}; or
(iv) C₇-C₂₀ aralkyl optionally substituted with from 1-10 R^{c}; or
(viii) —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}).
   R¹ can be:
(ii) C₁-C₁₀ alkyl optionally substituted with from 1-5 R^{a}; or
(iii) C₆-C₁₀ aryl optionally substituted with from 1-5 R^{b}; or
(iv) C₇-C₁₆ aralkyl optionally substituted with from 1-5 R^{c}; or
(viii) —C(O)NR^{g}R^{h}; —OC(O)NR^{g}R^{h}; —C(O)Rⁱ, —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)OR; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h}).
R¹ can be C₁-C₂₀ alkyl optionally substituted with from 1-10 R^{a} (e.g., C₁-C₁₀ alkyl optionally substituted with from 1-5 R^{a}; C₁-C₆ alkyl optionally substituted with from 1-3 R^{a}; or C₁-C₃ alkyl optionally substituted with from 1-2 R^{a}). R¹ can be CH₃. R¹ can be C₆-C₁₈ aryl, optionally substituted with from 1-10 R^{b} (e.g., C₆-C₁₀ aryl, optionally substituted with from 1-5 R^{b}; phenyl optionally substituted with 1, 2, 3, 4, or 5 R^{b}). R^{b} at each occurrence can be, independently, C₁-C₆ alkyl, C₁-C₆ haloalkyl, C₁-C₆ alkoxy, C₁-C₆ haloalkoxy, halo, NO₂, NR^{g}R^{h}, or cyano. R^{b} at each occurrence can be, independently, C₁-C₃ alkyl, C₁-C₃ haloalkyl, C₁-C₃ alkoxy, C₁-C₃ haloalkoxy, halo, NO₂, NH₂, or cyano). The C₁-C₃ haloalkyl can include 1, 2, 3, 4, or 5 halogens or can be C₁-C₃ perhaloalkyl, in which the halogen can be, for example, fluoro. R¹ can be phenyl.
R¹ can be C₇-C₂₀ aralkyl optionally substituted with from 1-10 R^{c} (e.g., C₇-C₁₂ aralkyl optionally substituted with from 1-5 R^{c}). R¹ can be benzyl. R¹ can be hydrogen.
R¹ can be —C(O)Rⁱ. For example, Rⁱ can be C₆-C₁₈ aryl or heteroaryl including 5-16 atoms, each of which is optionally substituted with from 1-10 R^{b}. Rⁱ can be phenyl or phenyl substituted with 1, 2, 3, 4, or 5 R^{b}. R^{b} at each occurrence can be, independently, C₁-C₆ alkyl, C₁-C₆ haloalkyl, C₁-C₆ alkoxy, C₁-C₆ haloalkoxy, halo, NO₂, NR^{g}R^{h}, or cyano.
   R² can be:
(i) C₆-C₁₈ aryl optionally substituted with from 1-10 R^{b}; or
(ii) C₇-C₂₀ aralkyl optionally substituted with from 1-10 R^{c}; or
(iii) C₃-C₂₀ cycloalkyl or C₃-C₂₀ halocycloalkyl, optionally substituted with from 1-10 R^{e}; or
(iv) C₃-C₂₀ cycloalkenyl optionally substituted with from 1-10 R^{f}.
R² can be C₆-C₁₈ aryl or heteroaryl including 5-16 atoms, each of which is optionally substituted with from 1-10 R^{b}.
R² can be C₆-C₁₈ aryl optionally substituted with from 1-10 R^{b} (e.g., C₆-C₁₀ aryl, optionally substituted with from 1-5 R^{b}; phenyl optionally substituted with from 1-5 R^{b}; phenyl optionally substituted with from 1-3 R^{b}). R² can be phenyl. R² can be phenyl substituted with 1, 2, 3, 4, or 5 R^{b}. R² can be phenyl substituted with 1, 2, 3, or 4 R^{b}. R² can be phenyl substituted with 1, 2, or 3 R^{b}. R² can be phenyl substituted with from 1 or 2 R^{b}. R² can be phenyl substituted with 1 R^{b}.
In some embodiments, when R² is C₆-C₁₈ aryl or heteroaryl including 5-16 atoms, each of which is optionally substituted with from 1-10 R^{b}; or C₆-C₁₈ aryl optionally substituted with from 1-10 R^{b}; or C₆-C₁₀ aryl, optionally substituted with from 1-5 R^{b}; or R² is phenyl substituted with 1, 2, 3, 4, or 5 R^{b}; or R² is phenyl substituted with 1, 2, 3, or 4 R^{b}; or R² is phenyl substituted with 1, 2, or 3 R^{b}; or R² is phenyl substituted with 1 or 2 R^{b}; or R² is phenyl substituted with 1 R^{b}, then R^{b} at each occurrence can be, independently:
(i) halo; NO₂; NR^{g}R^{h}; hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{b′}; C₇-C₂₀ aralkoxy or heteroaralkoxy including 6-20 atoms, each of which is optionally substituted with from 1-10 R^{c}; C₃-C₁₆ cycloalkoxy or C₃-C₁₆ halocycloalkoxy, each of which is optionally substituted with from 1-10 R^{e}; C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{a}; C₆-C₁₈ thioaryloxy or thioheteroaryloxy including 5-16 atoms, each of which is optionally substituted with from 1-10 R^{b′}; C₇-C₂₀ thioaralkoxy or thioheteroaralkoxy including 6-20 atoms, each of which is optionally substituted with from 1-10 R^{c}; C₃-C₂₀ thiocycloalkoxy or C₃-C₂₀ thiohalocycloalkoxy, each of which is optionally substituted with from 1-10 R^{e}; C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)NR^{g}R^{h}; —OC(O)NR^{g}R^{h}; —C(O)Rⁱ; —C(O)ORⁱ; —OC(O)Rⁱ; —C(O)SRⁱ; —SC(O)Rⁱ; —C(S)SRⁱ; —SC(S)Rⁱ; —NR^{j}C(O)Rⁱ; —NR^{j}C(O)ORⁱ; —NR^{j}C(O)NR^{g}R^{h}; —S(O)ₙR^{k}; —NR^{j}S(O)ₙRⁱ; —C(NR^{m})Rⁱ; or —P(O)(OR^{g})(OR^{h});
(ii) C₁-C₂₀ alkyl or C₁-C₂₀ haloalkyl, each of which is optionally substituted with from 1-10 R^{a}; or
(vi) C₇-C₂₀ aralkyl or heteroaralkyl including 6-20 atoms, each of which is optionally substituted with from 1-10 R^{c}; or
(vii) C₆-C₁₈ aryl or heteroaryl including 5-16 atoms, each of which is optionally substituted with from 1-10 R^{b′}; or
   R^{b} at each occurrence can be, independently:
(i) halo; NO₂; NR^{g}R^{h}; hydroxy; C₁-C₂₀ alkoxy optionally substituted with from 1-10 R^{a}; C₆-C₁₈ aryloxy or heteroaryloxy including 5-16 atoms, each of which is optionally substituted with from 1-10 R^{b′}; C₇-C₂₀ aralkoxy or heteroaralkoxy including 6-20 atoms, each of which is substituted with from 1-10 R^{c}; C₆-C₁₈ thioaryloxy or thioheteroaryloxy including 5-16 atoms, each of which is optionally substituted with from 1-10 R^{b′}; C₇-C₂₀ thioaralkoxy or thioheteroaralkoxy including 6-20 atoms, each of which is optionally substituted with from 1-10 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k}; or
(ii) C₁-C₂₀ alkyl or C₁-C₂₀ haloalkyl, each of which is optionally substituted with from 1-10 R^{a}; or
(vi) C₇-C₂₀ aralkyl or heteroaralkyl including 6-20 atoms, each of which is optionally substituted with from 1-10 R^{c}; or
(vii) C₆-C₁₈ aryl or heteroaryl including 5-16 atoms, each of which is optionally substituted with from 1-10 R^{b′}; or
   R^{b} at each occurrence can be, independently:
(i) halo; NO₂; NR^{g}R^{h}; hydroxy; C₁-C₁₀ alkoxy optionally substituted with from 1-5 R^{a}; C₆-C₁₄ aryloxy or heteroaryloxy including 5-14 atoms, each of which is optionally substituted with from 1-10 R^{b′}; C₇-C₂₀ aralkoxy or heteroaralkoxy including 6-20 atoms, each of which is substituted with from 1-10 R^{c}; C₆-C₁₄ thioaryloxy or thioheteroaryloxy including 5-14 atoms, each of which is optionally substituted with from 1-10 R^{b′}; C₇-C₂₀ thioaralkoxy or thioheteroaralkoxy including 6-20 atoms, each of which is optionally substituted with from 1-10 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k}; or
(ii) C₁-C₁₀ alkyl or C₁-C₁₀ haloalkyl, each of which is optionally substituted with from 1-5 R^{a}; or
(vi) C₇-C₁₆ aralkyl or heteroaralkyl including 6-16 atoms, each of which is optionally substituted with from 1-10 R^{c}; or
(vii) C₆-C₁₄ aryl or heteroaryl including 5-14 atoms, each of which is optionally substituted with from 1-10 R^{b′}; or
   R^{b} at each occurrence can be, independently:
(i) halo; NO₂; NR^{g}R^{h}; hydroxy; C₁-C₆ alkoxy optionally substituted with from 1-3 R^{a}; C₆-C₁₀ aryloxy or heteroaryloxy including 5-10 atoms, each of which is optionally substituted with from 1-5 R^{b′}; C₇-C₁₆ aralkoxy or heteroaralkoxy including 6-16 atoms, each of which is substituted with from 1-5 R^{c}; C₆-C₁₀ thioaryloxy or thioheteroaryloxy including 5-14 atoms, each of which is optionally substituted with from 1-5 R^{b′}; C₇-C₁₆ thioaralkoxy or thioheteroaralkoxy including 6-16 atoms, each of which is optionally substituted with from 1-5 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k}; or
(ii) C₁-C₆ alkyl or C₁-C₆ haloalkyl, each of which is optionally substituted with from 1-3 R^{a}; or
(vi) C₇-C₁₂ aralkyl or heteroaralkyl including 6-12 atoms, each of which is optionally substituted with from 1-5 R^{c}; or
(vii) C₆-C₁₀ aryl or heteroaryl including 5-10 atoms, each of which is optionally substituted with from 1-5 R^{b′}; or
   R^{b} at each occurrence can be, independently:
(i) halo; NO₂; NR^{g}R^{h}; hydroxy; C₁-C₃ alkoxy optionally substituted with from 1-2 R^{a}; C₆-aryloxy or heteroaryloxy including 5 or 6 atoms, each of which is optionally substituted with from 1-5 R^{b′}; C₇-C₁₂ aralkoxy or heteroaralkoxy including 6-12 atoms, each of which is substituted with from 1-5 R^{c}; C₆-thioaryloxy or thioheteroaryloxy including 5 or 6 atoms, each of which is optionally substituted with from 1-5 R^{b}; C₇-C₁₂ thioaralkoxy or thioheteroaralkoxy including 6-12 atoms, each of which is optionally substituted with from 1-5 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k}; or
(ii) C₁-C₃ alkyl or C₁-C₃ haloalkyl, each of which is optionally substituted with from 1-2 R^{a}; or
(vi) C₇-C₁₀ aralkyl or heteroaralkyl including 6-20 atoms, each of which is optionally substituted with from 1-5 R^{c}; or
(vii) phenyl or heteroaryl including 5 or 6 atoms, each of which is optionally substituted with from 1-5 R^{b}.
   R² can be: 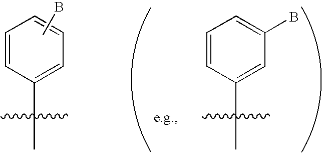
   wherein B is:
(i) halo; NO₂; NR^{g}R^{h}; hydroxy; C₁-C₂₀ alkoxy optionally substituted with from 1-10 R^{a}; C₆-C₁₈ aryloxy or heteroaryloxy including 5-16 atoms, each of which is optionally substituted with from 1-10 R^{b′}; C₇-C₂₀ aralkoxy or heteroaralkoxy including 6-20 atoms, each of which is substituted with from 1-10 R^{c}; C₆-C₁₈ thioaryloxy or thioheteroaryloxy including 5-16 atoms, each of which is optionally substituted with from 1-10 R^{b′}; C₇-C₂₀ thioaralkoxy or thioheteroaralkoxy including 6-20 atoms, each of which is optionally substituted with from 1-10 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k} or
(ii) C₁-C₂₀ alkyl or C₁-C₂₀ haloalkyl, each of which is optionally substituted with from 1-10 R^{a}; or
(iii) C₆-C₁₈ aryl or heteroaryl including 5-16 atoms, each of which is optionally substituted with from 1-10 R^{b′}; or
(iv) C₇-C₂₀ aralkyl or heteroaralkyl including 6-20 atoms, each of which is optionally substituted with from 1-10 R^{c}; or
(v) hydrogen; B can also be other than hydrogen, i.e., (i), (ii), (iii), or (iv). B can be hydrogen.
   B can be:
(i) halo; NO₂; NR^{g}R^{h}; hydroxy; C₁-C₁₀ alkoxy optionally substituted with from 1-5 R^{a}; C₆-C₁₄ aryloxy or heteroaryloxy including 5-14 atoms, each of which is optionally substituted with from 1-10 R^{b′}; C₇-C₂₀ aralkoxy or heteroaralkoxy including 6-20 atoms, each of which is substituted with from 1-10 R^{c}; C₆-C₁₄ thioaryloxy or thioheteroaryloxy including 5-14 atoms, each of which is optionally substituted with from 1-10 R^{b′}; C₇-C₂₀ thioaralkoxy or thioheteroaralkoxy including 6-20 atoms, each of which is optionally substituted with from 1-10 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k}; or
(ii) C₁-C₁₀ alkyl or C₁-C₁₀ haloalkyl, each of which is optionally substituted with from 1-5 R^{a}; or
(iii) C₇-C₁₆ aralkyl or heteroaralkyl including 6-20 atoms, each of which is optionally substituted with from 1-10 R^{c}; or
(iv) C₆-C₁₄ aryl or heteroaryl including 5-14 atoms, each of which is optionally substituted with from 1-10 R^{b}.
   B can be:
(i) halo; NO₂; NR^{g}R^{h}; hydroxy; C₁-C₆ alkoxy optionally substituted with from 1-3 R^{a}; C₆-C₁₀ aryloxy or heteroaryloxy including 5-10 atoms, each of which is optionally substituted with from 1-5 R^{b′}; C₇₋C₁₆ aralkoxy or heteroaralkoxy including 6-16 atoms, each of which is substituted with from 1-5 R^{c}; C₆-C₁₀ thioaryloxy or thioheteroaryloxy including 5-14 atoms, each of which is optionally substituted with from 1-5 R^{b′}; C₇-C₁₆ thioaralkoxy or thioheteroaralkoxy including 6-16 atoms, each of which is optionally substituted with from 1-5 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k}; or
(ii) C₁-C₆ alkyl or C₁-C₆ haloalkyl, each of which is optionally substituted with from 1-3 R^{a}; or
(iii) C₇-C₁₂ aralkyl or heteroaralkyl including 6-20 atoms, each of which is optionally substituted with from 1-5 R^{c}; or
(iv) C₆-C₁₀ aryl or heteroaryl including 5-10 atoms, each of which is optionally substituted with from 1-5 R^{b}.
   B can be:
(i) halo; NO₂; NR^{g}R^{h}; hydroxy; C₁-C₃ alkoxy optionally substituted with from 1-2 R^{a}; C₆-aryloxy or heteroaryloxy including 5 or 6 atoms, each of which is optionally substituted with from 1-5 R^{b′}; C₇-C₁₂ aralkoxy or heteroaralkoxy including 6-12 atoms, each of which is substituted with from 1-5 R^{c}; C₆-thioaryloxy or thioheteroaryloxy including 5 or 6 atoms, each of which is optionally substituted with from 1-5 R^{b′}; C₇-C₁₂ thioaralkoxy or thioheteroaralkoxy including 6-12 atoms, each of which is optionally substituted with from 1-5 R^{c}; cyano; —C(O)NR^{g}R^{h}; —C(O)Rⁱ; —NR^{j}C(O)Rⁱ; —NR^{j}C(O)NR^{g}R^{h}; or —S(O)ₙR^{k}; or
(ii) C₁-C₃ alkyl or C₁-C₃ haloalkyl, each of which is optionally substituted with from 1-2 R^{a}; or
(iii) C₇-C₁₀ aralkyl or heteroaralkyl including 6-20 atoms, each of which is optionally substituted with from 1-5 R^{c}; or
(iv) C₆-aryl or heteroaryl including 5 or 6 atoms, each of which is optionally substituted with from 1-5 R^{b}.
B can be hydroxy. B can be NH₂. B can be halo (e.g., fluoro or chloro). B can be C₁-C₆ alkoxy (e.g., OCH₃). B can be C₁-C₄ haloalkyl (e.g., CF₃). B can be —C(O)Rⁱ(e.g., formyl).
B can be C₁-C₆ alkyl, optionally substituted with 1 R^{a} (e.g., B can be a substituted CH₃ group). R^{a} can be NR^{g}R^{h}. For example, one of R^{g} and R^{h} can be hydrogen, and the other can be C₆-C₁₈ aryl or heteroaryl including 5-16 atoms, each of which can be optionally substituted with from 1-10 R^{b}. In some embodiments, one of R^{g} and R^{h} can be hydrogen, and the other can be a phenyl or napthyl group, each of which is optionally substituted with from 1-5 (e.g., 1-3) R^{b} (e.g., C₁-C₄ alkyl (e.g., CH₃) optionally substituted with 1 R^{a} (e.g., COOH)). For example, one of R^{g} and R^{h} can be hydrogen, and the other can be a phenyl ring in which an ortho position, a meta position, and the para position are each substituted with a combination of CH₃ and CH₂C(O)OH.
B can be —NR^{j}C(O)NR^{g}R^{h}. R^{j} can be hydrogen or C₁-C₆ alkyl (e.g., C₁-C₃ alkyl). R^{j} can be hydrogen. One of R^{g} and R^{h} can be hydrogen, and the other can be C₇-C₂₀ aralkyl or heteroaralkyl including 6-20 atoms, each of which is optionally substituted with from 1-10 R^{c}; or C₆-C₁₈ aryl or heteroaryl including 5-16 atoms, each of which is optionally substituted with from 1-10 R^{b}.
   For example, B can be: 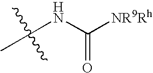
   One of R^{g} and R^{h} can be hydrogen, and the other can be C₇-C₂₀ aralkyl optionally substituted with from 1-10 R^{c}; or C₆-C₁₈ aryl optionally substituted with from 1-10 R^{b}. One of R^{g} and R^{h} can be hydrogen, and the other can be C₆-C₁₈ aryl optionally substituted with from 1-10 R^{b}. One of R^{g} and R^{h} can be hydrogen, and the other can be C₆-C₁₀ aryl optionally substituted with from 1-5 R^{b}. One of R^{g} and R^{h} can be hydrogen, and the other can be phenyl optionally substituted with from 1, 2, 3, 4,or 5 R^{b}. One of R^{g} and R^{h} can be hydrogen, and the other can be phenyl. One of R^{g} and R^{h} is hydrogen, and the other can be phenyl substituted with from 1, 2, 3, or 4 R^{b}. R^{b} at each occurrence can be, independently, halo; NO₂; hydroxy; C₁-C₁₀ alkoxy; cyano; —C(O)Rⁱ; C₁-C₁₀ alkyl; or C₁-C₁₀ haloalkyl (e.g., halo, NO₂, hydroxyl, C₁-C₆ alkoxy, cyano, —C(O)Rⁱ, C₁-C₆ alkyl, or C₁-C₆ haloalkyl; e.g., halo, NO₂, hydroxy; C₁-C₃ alkoxy, cyano, —C(O)Rⁱ, C₁-C₃ alkyl, or C₁-C₃ haloalkyl). The C₁-C₃ haloalkyl can include 1, 2, 3, 4, or 5 halogens or can be C₁-C₃ perhaloalkyl, in which the halogen can be, for example, fluoro).
   B can be:
(i-B) NR^{g}R^{h}, wherein one of R^{g} and R^{h} is hydrogen, and the other is C₇-C₂₀ aralkyl or heteroaralkyl including 6-20 atoms, each of which is optionally substituted with from 1-10 R^{c}; or C₆-C₁₈ aryl or heteroaryl including 5-16 atoms, each of which is optionally substituted with from 1-10 R^{b}; or
(ii-B) C₆-C₁₈ aryloxy or heteroaryloxy including 5-16 atoms, each of which is optionally substituted with from 1-10 R^{b′}; or C₇-C₂₀ aralkoxy or heteroaralkoxy including 6-20 atoms, each of which is optionally substituted with from 1-10 R^{c}; or
(iii-B) C₆-C₁₈ thioaryloxy or thioheteroaryloxy including 5-16 atoms, each of which is optionally substituted with from 1-10 R^{b′}; or C₇-C₂₀ thioaralkoxy or thioheteroaralkoxy including 6-20 atoms, each of which is optionally substituted with from 1-10 R^{c}; or
(vi-B) C₆-C₁₈ aryl or heteroaryl including 5-16 atoms, each of which is optionally substituted with from 1-10 R^{b′}; or C₇-C₂₀ aralkyl or heteroaralkyl including 6-20 atoms, each of which is optionally substituted with from 1-10 R^{c}.
   B can be:
(i-B′) NR^{g}R^{h}, wherein one of R^{g} and R^{h} is hydrogen, and the other is C₇-C₂₀ (e.g., C₇-C₁₆, C₇-C₁₂, C₇-C₁₀) aralkyl or heteroaralkyl including 6-20 (e.g., 6-14, 6-10) atoms, each of which is optionally substituted with from 1-10 (e.g., 1-5, 1-4, 1-3, 1-2, 1) R^{c};
(ii-B′) C₇-C₂₀ (e.g., C₇-C₁₆, C₇-C₁₂, C₇-C₁₀) aralkoxy or heteroaralkoxy including 6-20 (e.g., 6-14, 6-12, 6-10) atoms, each of which is optionally substituted with from 1-10 (e.g., 1-5, 1-4, 1-3, 1-2, 1) R^{c}; or
(iii-B′) C₇-C₂₀ (e.g., C₇-C₁₆, C₇-C₁₂, C₇-C₁₀) thioaralkoxy or thioheteroaralkoxy including 6-20 (e.g., 6-14, 6-12, 6-10) atoms, each of which is optionally substituted with from 1-10 (e.g., 1-5, 1-4, 1-3, 1-2, 1) R^{c}; or
(iv-B′) C₇-C₂₀ (e.g., C₇-C₁₆, C₇-C₁₂, C₇-C₁₀) aralkyl or heteroaralkyl including 6-20 (e.g., 6-14, 6-12, 6-10) atoms, each of which is optionally substituted with from 1-10 (e.g., 1-5, 1-4, 1-3, 1-2, 1) R^{c}.
In some embodiments, when B is (i-B), (ii-B), (iii-B), (iv-B), (i-B′), (ii-B′), (iii-B′), or (iv-B′), then R^{b}, R^{b′ and R}^{c} at each occurrence can each be, independently, halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ.
In some embodiments, when B is (i-B), (ii-B), (iii-B), (iv-B), (i-B′), (ii-B′), (iii-B′), or (iv-B′), then R^{b}, R^{b′ and R}^{c} at each occurrence can each be, independently, halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ.
In some embodiments, when B is (i-B), (ii-B), (iii-B), (iv-B), (i-B′), (ii-B′), (iii-B′), or (iv-B′), then R^{b}, R^{b′ and R}^{c} at each occurrence can each be, independently, halo; NO₂; hydroxy; C₁-C₆ alkoxy; C₁-C₆ haloalkoxy; cyano; —C(O)Rⁱ; C₁-C₆ alkyl or C₁-C₆ haloalkyl, each of which is optionally substituted with from 1-3 R^{a}; or —C(O)ORⁱ.
In some embodiments, when B is (i-B), (ii-B), (iii-B), (iv-B), (i-B′), (ii-B′), (iii-B′), or (iv-B′), then R^{b}, R^{b′ and R}^{c} at each occurrence can each be, independently, halo; NO₂; hydroxy; C₁-C₆ alkoxy; cyano; —C(O)Rⁱ; C₁-C₆ alkyl or C₁-C₆ haloalkyl, each of which is optionally substituted with from 1-3 R^{a}; or —C(O)ORⁱ.
In some embodiments, when B is (i-B), (ii-B), (iii-B), (iv-B), (i-B′), (ii-B′), (iii-B′), or (iv-B′), then R^{b}, R^{b} and R^{c} at each occurrence can each be, independently, halo; NO₂; hydroxy; C₁-C₃ alkoxy; C₁-C₃ haloalkoxy; cyano; —C(O)Rⁱ; C₁-C₄ alkyl or C₁-C₄ haloalkyl, each of which is optionally substituted with from 1-2 R^{a}; or —C(O)ORⁱ.
In some embodiments, when B is (i-B), (ii-B), (iii-B), (iv-B), (i-B′), (ii-B′), (iii-B′), or (iv-B′), then R^{b}, R^{b′ and R}^{c} at each occurrence can each be, independently, halo; NO₂; hydroxy; C₁-C₃ alkoxy; cyano; —C(O)Rⁱ; C₁-C₄ alkyl or C₁-C₄ haloalkyl, each of which is optionally substituted with from 1-2 R^{a}; or —C(O)ORⁱ.
In some embodiments, when B is (i-B), (ii-B), (iii-B), (iv-B), (i-B′), (ii-B′), (iii-B′), or (iv-B′), then R^{b}, R^{b′ and R}^{c} at each occurrence can each be, independently, halo; NO₂; hydroxy; C₁-C₃ alkoxy; C₁-C₃ haloalkoxy; cyano; —C(O)Rⁱ; C₁-C₄ alkyl; C₁-C₄ haloalkyl; C₁-C₄ alkyl substituted with from 1-2 R^{a}; —C(O)OH; or —C(O)OCH₃.
In some embodiments, when B is (i-B), (ii-B), (iii-B), (iv-B), (i-B′), (ii-B′), (iii-B′), or (iv-B′), then R^{b}, R^{b′ and R}^{c} at each occurrence can each be, independently, halo; NO₂; hydroxy; C₁-C₃ alkoxy; cyano; —C(O)Rⁱ; C₁-C₄ alkyl; C₁-C₄ haloalkyl; C₁-C₄ alkyl substituted with from 1-2 R^{a}; —C(O)OH; or —C(O)OCH₃.
In some embodiments, when B is (i-B), (ii-B), (iii-B), (iv-B), (i-B′), (ii-B′), (iii-B′), or (iv-B′), R^{a} can be —C(O)OH or —C(O)OCH₃; and/or C₁-C₄ haloalkyl can be C₁-C₄ perfluoroalkyl.
   B can be: 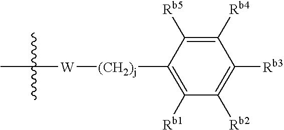
   wherein:
W can be NR^{j}, O, S, or is absent;
j can be 0, 1, 2, 3, 4, or 5; and
each of R^{b1}, R^{b2}, R^{b3}, R^{b4}, and R^{b5} is, independently, hydrogen, halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ.
Each of R^{b1}, R^{b2}, R^{b3}, R^{b4}, and R^{b5} can be, independently, hydrogen, halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ.
W can be NR^{j}, O, or S. R^{j} can be hydrogen or C₁-C₆ alkyl (e.g., C₁-C₃ alkyl). R^{j} can be hydrogen. j can be 0 or 1 (e.g., 1).
R^{b1}, R^{b2}, R^{b3}, R^{b4}, and R^{b5} can each be, independently, hydrogen; halo; NO₂; hydroxy; C₁-C₆ alkoxy; C₁-C₆ haloalkoxy; cyano; —C(O)Rⁱ; C₁-C₆ alkyl or C₁-C₆ haloalkyl, each of which is optionally substituted with from 1-3 R^{a}; or —C(O)ORⁱ.
R^{b1}, R^{b2}, R^{b3}, R^{b4}, and R^{b5} can each be, independently, hydrogen; halo; NO₂; hydroxy; C₁-C₆ alkoxy; cyano; —C(O)Rⁱ; C₁-C₆ alkyl or C₁-C₆ haloalkyl, each of which is optionally substituted with from 1-3 R^{a}; or —C(O)ORⁱ.
R^{b1}, R^{b2}, R^{b3}, R^{b4}, and R^{b5} can each be, independently, hydrogen; halo; NO₂; hydroxy; C₁-C₃ alkoxy; C₁-C₃ haloalkoxy; cyano; —C(O)Rⁱ; C₁-C₄ alkyl or C₁-C₄ haloalkyl, each of which is optionally substituted with from 1-2 R^{a}; or —C(O)ORⁱ.
R^{b1}, R^{b2}, R^{b3}, R^{b4}, and R^{b5} can each be, independently, hydrogen; halo; NO₂; hydroxy; C₁-C₃ alkoxy; cyano; —C(O)Rⁱ; C₁-C₄ alkyl or C₁-C₄ haloalkyl, each of which is optionally substituted with from 1-2 R^{a}; or —C(O)ORⁱ.
R^{b1}, R^{b2}, R^{b3}, R^{b4}, and R^{b5} can each be, independently, hydrogen; F; Cl; Br; OH; OCH₃; OCF₃; —C(O)(morpholino); CH₃; CH₃ substituted with from 1-2 R^{a} (e.g., —C(O)OH or —C(O)OCH₃); CF₃; —C(O)OH; or —C(O)OCH₃.
R^{b1}, R^{b2}, R^{b3}, R^{b4}, and R^{b5} can each be, independently, hydrogen; F; Cl; Br; OH; OCH₃; —C(O)(morpholino); CH₃; CH₃ substituted with from 1-2 R^{a} (e.g., —C(O)OH or —C(O)OCH₃); CF₃; —C(O)OH; or —C(O)OCH₃.
One of R^{b1}, R^{b2}, R^{b3}, R^{b4}, or R^{b5} (e.g., R^{b3}) can be halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and the other four can be hydrogen.
One of R^{b1}, R^{b2}, R^{b3}, R^{b4}, or R^{b5} (e.g., R^{b3}) can be halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and the other four can be hydrogen. One of R^{b1}, R^{b2}, R^{b3}, R^{b4}, or R^{b5} can be C₁-C₁₀ haloalkoxy (e.g., OCF₃), and the other four can be hydrogen.
R^{b3} can be C₁-C₄ alkyl substituted with from 1 R^{a}. R^{a} can be C(O)ORⁱ. Rⁱ can be hydrogen or C₁-C₄ alkyl (e.g., CH₃). R^{b3} can be —CH₂C(O)OH, —CH₂C(O)OCH₃, —C(CH₃)₂C(O)OH, or —C(CH₃)₂C(O)OCH₃. R^{b3} can be —C(O)OR (e.g., COOH).
R^{b1} can be C₁-C₆ haloalkoxy (e.g., OCF₃). R^{b1} can be halo (e.g., chloro).
R^{b2} can be C₁-C₄ haloalkyl (e.g., CF₃); or —C(O)ORⁱ (e.g., COOH); or —C(O)Rⁱ (e.g., —C(O)(morpholino)).
Two of R^{b1}, R^{b2}, R^{b3}, R^{b4}, or R^{b5} can each be, independently, halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and the other three are hydrogen.
Two of R^{b1}, R^{b2}, R^{b3}, R^{b4}, or R^{b5} can each be, independently, halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and the other three are hydrogen. One or both of R^{b1}, R^{b2}, R^{b3}, R^{b4}, or R^{b5} can be C₁-C₁₀ haloalkoxy (e.g., OCF₃), and the others can be hydrogen.
R^{b1} and R^{b4} can each be, independently, halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and each of R^{b2}, R^{b3}, and R^{b5} is hydrogen.
R^{b1} and R^{b4} can each be, independently, halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and each of R^{b2}, R^{b3}, and R^{b5} is hydrogen.
R^{b1} and R^{b4} can each be, independently, halo; C₁-C₆ alkyl; C₁-C₄ haloalkyl; or C₁-C₆ alkoxy; and each of R^{b2}, R^{b3}, and R^{b5} is hydrogen.
R^{b1} and R^{b4} can both be C₁-C₄ alkyl (e.g., CH₃), and each of R^{b2}, R^{b3}, and R^{b5} can be hydrogen.
R^{b1} and R^{b4} can both be C₁-C₄ haloalkyl (e.g., CF₃), and each of R^{b2}, R^{b3}, and R^{b5} can be hydrogen.
R^{b1} can be C₁-C₄ haloalkyl (e.g., CF₃), R^{b4} can be halo (e.g., fluoro or chloro), and each of R^{b2}, R^{b3}, and R^{b5} can be hydrogen.
One of R^{b1} and R^{b4} can be halo (e.g., bromo), and the other can be C₁-C₆ alkoxy (e.g., OCH₃); and each of R^{b2}, R^{b3}, and R^{b5} can be hydrogen.
R^{b1} can be halo (e.g, fluoro or chloro); R^{b4} can be C₁-C₄ haloalkyl (e.g., CF₃) or halo (e.g., fluoro, chloro, or bromo); and each of R^{b2}, R^{b3}, and R^{b5} can be hydrogen.
R^{b1} and R^{b2} can each be, independently, halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and each of R^{b3}, R^{b4}, and R^{b5} is hydrogen.
R^{b1} and R^{b2} can each be, independently, halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and each of R^{b3}, R^{b4}, and R^{b5} is hydrogen.
R^{b1} and R^{b2} can both be C₁-C₄ alkyl (e.g., CH₃), and each of R^{b3}, R^{b4}, and R^{b5} can be hydrogen.
R^{b1} can be halo (e.g., fluoro or chloro), R^{b2} can be C₁-C₄ haloalkyl (e.g., CF₃), and each of R^{b3}, R^{b4}, and R^{b5} can be hydrogen.
R^{b2} and R^{b3} can each be, independently, halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and each of R^{b1}, R^{b2}, and R^{b5} is hydrogen.
R^{b2} and R^{b3} can each be, independently, halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and each of R^{b1}, R^{b4}, and R^{b5} is hydrogen.
R^{b2} and R^{b3} can each be, independently, halo; C₁-C₆ alkoxy; or —C(O)ORⁱ; and each of R^{b1}, R^{b4}, and R^{b5} is hydrogen.
R^{b2} and R^{b3} can both be halo (e.g., chloro), and each of R^{b1}, R^{b2}, and R^{b5} can be hydrogen.
R^{b2} and R^{b3} can each be, independently, C₁-C₆ alkoxy (e.g., OCH₃); or —C(O)ORⁱ (e.g., COOH); and each of R^{b1}, R^{b4}, and R^{b5} can be hydrogen.
R^{b1} and R^{b5} can each be, independently, halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and each of R^{b2}, R^{b3}, and R^{b4} is hydrogen. For example, R^{b1} and R^{b5} can both be halo (e.g., chloro), and each of R^{b2}, R^{b3}, and R^{b4} can be hydrogen.
R^{b1} and R^{b3} can each be, independently, halo; NO₂; hydroxy; C₁-C₁₀ alkoxy; C₁-C₁₀ haloalkoxy; cyano; —C(O)Rⁱ; C₁-C₁₀ alkyl or C₁-C₁₀ haloalkyl, each of which is optionally substituted with from 1-5 R^{a}; or —C(O)ORⁱ; and each of R^{b2}, R^{b4}, and R^{b5} is hydrogen. For example, R^{b1} can be halo (e.g., chloro), R^{b3} can be —C(O)ORⁱ (e.g., COOH), and each of R^{b2}, R^{b4}, and R^{b5} can be hydrogen.
Each of R^{b1} R^{b2} R^{b3} R^{b4} and R^{b5} can be hydrogen.
Each of R^{b1} R^{b2} R^{b3} R^{b4} and R^{b5} can be other than hydrogen.
When B is as described in (i-B), (ii-B), (iii-B), (iv-B), (i-B′), (ii-B′), (iii-B′), (iv-B′)), B can also be W—(CH₂)ⱼ-(bicyclic or tricyclic aryl) or W—(CH₂)ⱼ-(heteroaryl), in which W and j can be as described elsewhere.
B can be —NH—CH₂-naphthyl (e.g., the methylene group can be attached to the 1 or 2 position of the naphthyl ring, and the naphthyl ring can optionally be substituted in one or more positions, e.g., with 1-5, 1-4, 1-3, 1-2, or 1 R^{c}).
In certain embodiments, B can be —NH—CH₂-indolyl or —O—CH₂-indolyl (e.g., the methylene group can be attached to the 2 or 7 position of the indole ring, and the indole ring can be optionally substituted in one or more positions, e.g., with 1-5, 1-4, 1-3, 1-2, or 1 R^{c}, e.g., at the 1-position with CH₃ and/or at the 5-position with halo (e.g., fluoro) and/or at the 3-position with COOR (e.g., COOH).
In certain embodiments, B can be —NH—CH₂-benzothienyl (e.g., the methylene group can be attached to the 2 or 3 position of the benzothienyl ring, and the benzothienyl ring can be optionally substituted in one or more positions, e.g., with 1-5, 1-4, 1-3, 1-2, or 1 R^{c}, e.g., at the 3-position with C₁-C₆ alkyl (e.g., CH₃) or at the 4-position with C₁-C₄ haloalkyl (e.g., CF₃)).
B can be —C(O)NR^{g}R^{h}; —C(O)Rⁱ; —NR^{j}C(O)Rⁱ; —NR^{j}C(O)NR^{g}R^{h}; or —S(O)ₙR^{k}. R^{j} can be hydrogen or C₁-C₆ alkyl (e.g., C₁-C₃ alkyl). R^{j} can be hydrogen. Each of Rⁱ and R^{k} can be, independently, C₆-C₁₈ aryl or heteroaryl including 5-16 atoms, each of which is optionally substituted with from 1-10 R^{b′}; or C₇-C₂₀ aralkyl or heteroaralkyl including 6-20 atoms, each of which is optionally substituted with from 1-10 R^{c}. Each of Rⁱ and R^{k} can be, independently, C₆-C₁₈ aryl optionally substituted with from 1-10 R^{b′}; or C₇-C₂₀ aralkyl optionally substituted with from 1-10 R^{c} (R^{b′ and R}^{c} at each occurrence can each be, independently, halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ). One of R^{g} or R^{h} can be hydrogen, and the other can be C₆-C₁₈ aryl or heteroaryl including 5-16 atoms, each of which is optionally substituted with from 1-10 R^{b′}; or C₇-C₂₀ aralkyl or heteroaralkyl including 6-20 atoms, each of which is optionally substituted with from 1-10 R^{c}. One of R^{g} or R^{h} can be hydrogen, and the other can be C₆-C₁₈ aryl optionally substituted with from 1-10 R^{b′}; or C₇-C₂₀ aralkyl optionally substituted with from 1-10 R^{c} (R^{b′ and R}^{c} at each occurrence are each, independently, halo; NO₂; hydroxy; C₁-C₁₀ alkoxy; cyano; —C(O)Rⁱ; C₁-C₁₀ alkyl or C₁-C₁₀ haloalkyl, each of which is optionally substituted with from 1-5 R^{a}; or —C(O)ORⁱ).
R² can be ortho or para monosubstituted phenyl (e.g., 2-fluoro, 4-fluorophenyl, 4-trifluoromethylphenyl). R² can be disubstituted phenyl (e.g., 3,4-dihalophenyl, e.g., 3-chloro-4-fluorophenyl).
Each of R³, R⁴ and R⁵ can be, independently, hydrogen or halo. Each of R³, R⁴ and R⁵ can be hydrogen.
R⁶ can be halo or C₁-C₁₀ alkyl, or C₁-C₁₀ haloalkyl; R⁶ can be halo or C₁-C₆ alkyl, or C₁-C₆ haloalkyl; R⁶ can be halo or C₁-C₃ alkyl, or C₁-C₃ haloalkyl.
R⁶ can be C₁-C₁₀ (e.g., C₁-C₆ or C₁-C₃) alkyl. R⁶ can be CH₃.
R⁶ can be C₁-C₁₀ (e.g., C₁-C₆ or C₁-C₃) haloalkyl. R⁶ can be CF₃.
R⁶ can be halo (e.g., bromo or chloro, preferably chloro).
R⁶ can be hydrogen.

Also disclosed in U.S. patent application Ser. No. 11/365,750, and useful herein, are compounds having formula (III): 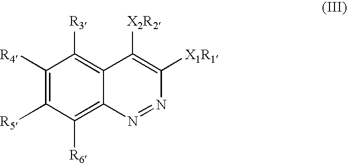
in which:

X₁ can be a bond, C₁ to C₅ alkyl, —C(O)—, —C(═CR₈R₉)—, —O—, —S(O)ₜ—, —NR₈—, —CR₈R₉—, —CHR₂₃, —CR₈(OR₉)—, —C(OR₈)₂—, —CR₈(OC(O)R₉)—, —C═NOR₉—, —C(O)NR₈—, —CH₂O—, —CH₂S—, —CH₂NR₈—, —OCH₂—, —SCH₂—, —NR₈CH₂—, or

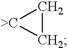

R_{1′} can be H, C₁ to C₆ alkyl, C₂ to C₆ alkenyl, C₂ to C₆ alkynyl, C₃ to C₆ cycloalkyl, —CH₂OH, C₇ to C₁₁ arylalkyl, phenyl, naphthyl, C₁ to C₃ perfluoroalkyl, CN, C(O)NH₂, CO₂R₁₂ or phenyl substituted independently by one or more of the groups independently selected from C₁ to C₃ alkyl, C₂ to C₄ alkenyl, C₂ to C₄ alkynyl, C₁ to C₃ alkoxy, C₁ to C₃ perfluoroalkyl, halogen, —NO₂, —NR₈R₉, —CN, —OH, and C₁ to C₃ alkyl substituted with 1 to 5 fluorines, or

R_{1′} can be a heterocycle selected from the group consisting of pyridine, thiophene, benzisoxazole, benzothiophene, oxadiazole, pyrrole, pyrazole, and furan, each of which may be optionally substituted with one to three groups independently selected from C₁ to C₃ alkyl, C₁ to C₃ alkoxy, C₁ to C₃ perfluoroalkyl, halogen, —NO₂, —NR₈R₉, —CN, and C₁ to C₃ alkyl substituted with 1 to 5 fluorines;

X₂ can be a bond or —CH₂—;

R_{2′} can be phenyl, naphthyl, or phenyl or naphthyl substituted independently by one to four groups independently selected from C₁ to C₃ alkyl, hydroxy, phenyl, acyl, halogen, —NH₂, —CN, —NO₂, C₁ to C₃alkoxy, C₁ to C₃ perfluoroalkyl, C₁ to C₃ alkyl substituted with 1 to 5 fluorines, NR₁₄R₁₅, —C(O)R₁₀, —C(O)NR₁₀R₁₁, —C(O)NR₁₁A, —C≡CR₈, —CH═CHR₈, —W′A, —C≡—CA, —CH═CHA, —W′YA, —W′YNR₁₁-A, —W′YR₁₀, —W′Y(CH₂)ⱼA, —W′CHR₁₁(CH₂)ⱼA, —W′(CH₂)ⱼA, —W′(CH₂)ⱼR₁₀, —CHR₁₁W′(CH₂)ⱼR₁₀, —CHR₁₁W′(CH₂)ⱼA, —CHR₁₁NR₁₂YA, —CHR₁₁NR₁₂YR₁₀, pyrrole, —W′(CH₂)ⱼA(CH₂)ₖD(CH₂)ₚZ, —W′(CR₁₈R₁₉)A(CH₂)ₖD(CH₂)ₚZ, —(CH₂)ⱼW′A(CH₂)ₖD(CH₂)ₚZ, —CH═CHA(CH₂)ₖD(CH₂)ₚZ, —C≡CA(CH₂)ₖD(CH₂)ₚZ, —W′(CH₂)ⱼC≡CA(CH₂)ₖD(CH₂)ₚZ, and —W′(CH₂)ⱼZ, or

R_{2′} can be a heterocycle selected from pyridine, pyrimidine, thiophene, furan, benzothiophene, indole, benzofuran, benzimidazole, benzothiazole, benzoxazole, and quinoline, each of which may be optionally substituted with one to three groups independently selected from C₁ to C₃ alkyl, C₁ to C₃ alkoxy, hydroxy, phenyl, acyl, halogen, —NH₂, —CN, —NO₂, C₁ to C₃ perfluoroalkyl, C₁ to C₃ alkyl substituted with 1 to 5 fluorines, —C(O)R₁₀, —C(O)NR₁₀R₁₁, —C(O)NR₁₁A, —C≡CR₈, —CH═CHR₈, —W′A, —C≡CA, —CH═CHA, —W′YA, —W′YR₁₀, —W′Y(CH₂)ⱼA, —W′(CH₂)ⱼA, —W′(CH₂)ⱼR₁₀, —CHR₁₁W′(CH₂)ⱼR₁₀, —CHR₁₁W′(CH₂)ⱼA, —CHR₁₁NR₁₂YA, —CHR₁₁ NR₁₂YR₁₀, —W′CHR₁₁(CH₂)ⱼA, —W′(CH₂)ⱼA(CH₂)ₖD(CH₂)ₚZ, —W′(CR₁₈R₁₉)A(CH₂)ₖD(CH₂)ₚZ, —(CH₂)ⱼW′A(CH₂)ₖD(CH₂)ₚZ, —CH═CHA(CH₂)ₖD(CH₂)ₚZ, —C≡CA(CH₂)ₖD(CH₂)ₚZ, —W′(CH₂)ⱼC≡CA(CH₂)ₖD(CH₂)ₚZ, and —W′(CH₂)ⱼZ;

W′ can be a bond, —O—, —S—, —S(O)—, —S(O)2—, —NR₁₁—, or —N(COR₁₂)—;

Y can be —CO—, —S(O)2—, —CONR₁₃, —CONR₁₃CO—, —CONR₁₃SO₂—, —C(NCN)—, —CSNR₁₃, —C(NH)NR₁₃, or —C(O)O—;

j can be 0 to 3; k can be 0 to 3; t can be 0 to 2;

D can be a bond, —CH═CH—, —C≡C—, —C═, —C(O)—, phenyl, —O—, —NH—, —S—, —CHR₁₄—, —CR₁₄R₁₅—, —OCHR₁₄—, —OCR₁₄R₁₅—, or —CH(OH)CH(OH)—;

p can be 0 to 3;

Z can be —CO₂R₁₁, —CONR₁₀R₁₁, —C(═NR₁₀)NR₁₁R₁₂, —CONH₂NH₂, —CN, —CH₂OH, —NR₁₆R₁₇, phenyl, CONHCH(R₂₀)COR₁₂, phthalimide, pyrrolidine-2,5-dione, thiazolidine-2,4-dione, tetrazolyl, pyrrole, indole, oxazole, 2-thioxo-1,3-thiazolinin-4-one, C₁ to C₇ amines, C₃ to C₇ cyclic amines, or C₁ to C₃ alkyl substituted with one to two OH groups; wherein said pyrrole is optionally substituted with one or two substituents independently selected from the group consisting of —CO₂CH₃, —CO₂H, —COCH₃, —CONH₂ and —CN; wherein said C₁ to C₇ amines are optionally substituted with one to two substituents independently selected from the group consisting of —OH, halogen, —OCH₃, and —C≡CH; wherein said phenyl is optionally substituted with CO₂R₁₁, and wherein said C₃ to C₇ cyclic amines are optionally substituted with one or two substituents independently selected from the group consisting of —OH —CH₂OH, C₁ to C₃ alkyl, —CH₂OCH₃, —CO₂CH₃, and —CONH₂, and wherein said oxazole is optionally substituted with CH₂CO₂R₁₁;

A can be phenyl, naphthyl, tetrahydronaphthyl, indan or biphenyl, each of which may be optionally substituted by one to four groups independently selected from halogen, C₁ to C₃ alkyl, C₂ to C₄ alkenyl, C₂ to C₄ alkynyl, acyl, hydroxy, halogen, —CN, —NO₂, —CO₂R₁₁, —CH₂CO₂R₁₁, phenyl, C₁ to C₃ perfluoroalkoxy, C₁ to C₃ perfluoroalkyl, —NR₁₀R₁₁, —CH₂NR₁₀R₁₁, —SR₁₁, C₁ to C₆ alkyl substituted with 1 to 5 fluorines, C₁ to C₃ alkyl substituted with 1 to 2 —OH groups, C₁ to C₆ alkoxy optionally substituted with 1 to 5 fluorines, or phenoxy optionally substituted with 1 to 2 CF₃ groups; or

A can be a heterocycle selected from pyrrole, pyridine, pyridine-N-oxide, pyrimidine, pyrazole, thiophene, furan, quinoline, oxazole, thiazole, imidazole, isoxazole, indole, benzo[1,3]-dioxole, benzo[1,2,5]-oxadiazole, isochromen-1-one, benzothiophene, benzofuran, 2,3-dihydrobenzo[1,4]-dioxine, bitheinyl, quinazolin-2,4-91,3H]dione, and 3-H-isobenzofuran-1-one, each of which may be optionally substituted by one to three groups independently selected from halogen, C₁ to C₃ alkyl, acyl, hydroxy, —CN, —NO₂, C₁ to C₃ perfluoroalkyl, —NR₁₀R₁₁, —CH₂NR₁₀R₁₁, —SR₁₁, C₁ to C₃ alkyl substituted with 1 to 5 fluorines, and C₁ to C₃ alkoxy optionally substituted with 1 to 5 fluorines;

R_{3′}, R_{4′}, and R_{5′} can each be, independently, —H or —F; R_{6′} can be hydrogen, C₁ to C₄ alkyl, C₁ to C₄ perfluoroalkyl, halogen, —NO₂, —CN, phenyl or phenyl substituted with one or two groups independently selected from halogen, C₁ to C₂ alkyl and OH;

each R₈ can be independently —H, or C₁ to C₃ alkyl;

each R₉ can be independently —H, or C₁ to C₃ alkyl;

each R₁₀ can be independently —H, C₁ to C₇ alkyl, C₃ to C₇ alkenyl, C₃ to C₇ alkynyl, C₃ to C₇ cycloalkyl, —CH₂CH₂OCH₃, 2-methyl-tetrahydro-furan, 2-methyl-tetrahydro-pyran, 4-methyl-piperidine, morpholine, pyrrolidine, or phenyl optionally substituted with one or two C₁ to C₃ alkoxy groups, wherein said C₁ to C₇ alkyl is optionally substituted with 1, 2 or 3 groups independently selected from C₁ to C₃ alkoxy, C₁ to C₃ thioalkoxy and CN;

each R₁₁ can be independently —H, C₁ to C₃ alkyl or R₂₂;

or R₁₀ and R₁₁, when attached to the same atom, together with said atom can form:

(i) a 5 to 7 membered saturated ring, optionally substituted by 1 to 2 groups independently selected from C₁ to C₃ alkyl, OH and C₁-C₃ alkoxy; or

(ii) a 5 to 7 membered ring containing 1 or 2 heteroatoms, optionally substituted by 1 to 2 groups independently selected from C₁ to C₃ alkyl, OH and C₁-C₃ alkoxy;

each R₁₂ can be independently —H, or C₁ to C₃ alkyl;

each R₁₃ can be independently —H, or C₁ to C₃ alkyl;

each R₁₄ and R₁₅ can be, independently, C₁ to C₇ alkyl, C₃ to C₈ cycloalkyl, C₂ to C₇ alkenyl, C₂ to C₇ alkynyl, —OH, —F, C₇ to C₁₄ arylalkyl, where said arylalkyl is optionally substituted with 1 to 3 groups independently selected from NO₂, C₁ to C₆ alkyl, C₁ to C₃ perhaloalkyl, halogen, CH₂CO₂R₁₁, phenyl and C₁ to C₃ alkoxy, or R₁₄ and R₁₅ together with the atom to which they are attached can form a 3 to 7 membered saturated ring;

each R₁₆ and R₁₇ can be, independently, hydrogen, C₁ to C₃ alkyl, C₁ to C₃ alkenyl, C₁ to C₃ alkynyl, phenyl, benzyl or C₃ to C₈ cycloalkyl, wherein said C₁ to C₃ alkyl is optionally substituted with one OH group, and wherein said benzyl is optionally substituted with 1 to 3 groups selected from C₁ to C₃ alkyl and C₁ to C₃ alkoxy; or

R₁₆ and R₁₇, together with the atom to which they are attached, can form a 3 to 8 membered heterocycle which is optionally substituted with one or two substituents independently selected from the group consisting of C₁ to C₃ alkyl, —OH, CH₂OH, —CH₂OCH₃, —CO₂CH₃, and —CONH₂;

each R₁₈ and R₁₉ can be, independently, C₁ to C₃ alkyl;

each R₂₀ can be independently H, phenyl, or the side chain of a naturally occurring alpha amino acid;

each R₂₂ can be independently arylalkyl optionally substituted with CH₂COOH; and

each R₂₃ can be phenyl; a compound of formula (VI) can be a salt or prodrug thereof (e.g., a pharmaceutically acceptable salt or prodrug).

Additionally, compounds disclosed in co-owned, copending U.S. Patent Application Publication No. 2006/0030612 are useful in the therapeutic or pharmaceutical compositions disclosed herein. Compounds disclosed therein include those having formula (IVa) or (IVb): 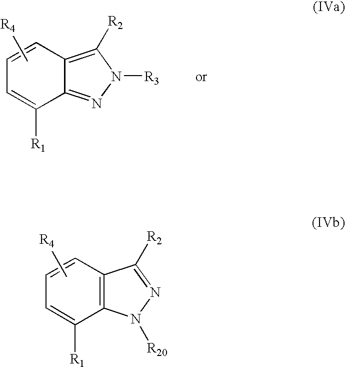
wherein:
R₁ is C₁₋₆ alkyl, CN, CO₂R₅, C(O)R₅, C₂₋₆ alkenyl, C₃₋₈ cycloalkenyl, C₂₋₆ alkynyl, NR₅R₆, C(O)NR₅R₆, phenyl, thiophene, C₁₋₃ alkoxy, halogen, or S(O)ₖR₅; wherein: said C₁₋₆ alkyl is optionally substituted with from 1 to 7 substituents independently selected from the group consisting of halogen and OH;
k is 0, 1 or2;
each R₅ and each R₆ is independently H, C₁₋₆ alkyl, C₃₋₈ cycloalkyl, S(O)₂-alkyl or arylalkyl; or

each R₅ and each R₆, together with the nitrogen atom to which they are attached, form independently:
a) a 3 to 7 membered saturated ring that is optionally substituted with C₁₋₃ alkyl, CH₂OH, or C(═O)NH₂; or
b) a 3 to 7 membered ring containing in its backbone one or two additional heteroatoms that is optionally substituted with up to three substituents independently selected from the group consisting of ═O, C₁₋₃ alkyl, COC₁₋₆ alkyl, and CO₂C₁₋₆ alkyl;
provided that when R₁ is S(O)ₖR₅, then said R₅ of said S(O)ₖR₅ is not S(O)₂-alkyl;
R₂ is C₃₋₈ alkyl, C₃₋₈ cycloalkyl, C₂₋₈ alkenyl, C₃₋₈ cycloalkenyl, C₂₋₈ alkynyl, NR₇R₈, aryl, arylalkyl, heteroaryl, heteroarylalkyl or heterocycloalkyl, wherein said C₃₋₈ alkyl, said C₃₋₈ cycloalkyl and said arylalkyl are each optionally substituted with up to four substituents independently selected from the group consisting of halogen, CN and OR₇, and wherein said heteroaryl is optionally substituted with YD; or
R₂ is phenyl substituted with up to four substituents independently selected from the group consisting of C₁₋₃ alkyl, C₂₋₈ alkenyl, C₂₋₈ alkynyl, C₁₋₃ alkoxy, C₃₋₈ cycloalkyl, halogen, OH, CH₂OH, CN, NR₇R₈, N(R₇)C(O)NR₅R₆, S(O)ₘR₇, phenyl, NO₂, C(O)R₇, OC(O)R₇, C(O)NR₇R₈, C(O)NR₇D and YD, providing any OH group present is not in the para position; wherein:
said C₁₋₃ alkyl and said C₁₋₃ alkoxy are each optionally substituted with from 1 to 7 fluorine atoms; m is 0 to 2; and R₅ and R₆ are as previously defined;
each R₇ and each R₈ is independently H or C₁₋₃ alkyl; or
each R₇ and each R₈, together with the N atom to which they are attached, form independently:
a) a 3 to 7 membered saturated ring which is optionally substituted with C₁₋₃ alkyl, CO₂R₁₄, CH₂CO₂R₁₄, OCH₂CO₂R₁₄, CH₂OCH₂CO₂R₁₄, C(O)NR₁₄R₁₅, CH₂OH, or CH₂CH₂OH; or
b) a 3 to 7 membered ring containing in its backbone one or two additional heteroatoms that is optionally substituted with CH₂CO₂R₁₄; wherein R₁₄ and R₁₅ are each independently H or C₁₋₃ alkyl;
Y is a bond, CH₂, CH₂CH₂, C₂₋₄ alkynylenyl, —O—, CH₂OCH₂, OCH₂, CH₂O, —N(R₇)—, —N(COR₇)—, S(O)ⱼ, —N(R₇)CH₂—, —N(R₇)CONR₈—, —N(COR₇)CH₂—, S(O)ⱼCH₂, —CH₂N(R₇)CH₂—, —CH₂N(COR₇)CH₂—, —OCH₂O—, —C(R₇)(CO₂R₈)— or —CH₂S(O)ⱼCH₂—; wherein R₇ and R₈ are as previously defined; and j is 0, 1 or 2;
D is tetrahydronaphthalene, tetrahydronaphthalol, tetralone, naphthalene, anthracene, benzyl or phenyl, each of which is optionally substituted with up to five independently selected R groups;
each R is independently selected from the group consisting of C₁₋₆ alkyl, C₁₋₃ alkoxy, halogen, —C(═O)H, —C(O)—C₁₋₃ alkyl, CH₂OH, CN, NH₂, NO₂, C₂₋₄ alkenyl, C₂₋₄ alkynyl, S(O)ⱼR₉, and WX; wherein said C₁₋₆ alkyl and said C₁₋₃ alkoxy are each optionally substituted with from 1 to 7 fluorine atoms; and j is 0, 1 or 2; or
D is a heterocycloalkyl, heterocycloalkylalkyl, heteroarylalkyl, heteroaryl or arylalkyl group, each of which is optionally substituted with up to four independently selected R^{a} groups;
each R^{a} is independently selected from the group consisting of C₁₋₈ alkyl, phenyl, benzyl, C₃₋₈ cycloalkyl C₇₋₁₁ arylalkyl, C₁₋₃ alkoxy, halogen, —C(═O)H, —C(O)—C₁₋₃ alkyl, CH₂OH, CN, NO₂, NH₂, OH, ═O, C₂₋₆ alkenyl, C₂₄ alkynyl, S(O)ⱼR₉ and WX;
wherein said C₁₋₈ alkyl, said C₂₋₆ alkenyl, said C₂₋₄ alkynyl and said C₁₋₃ alkoxy are each optionally substituted with from 1 to 7 fluorine atoms; and
j is 0, 1 or 2;
W is a bond, —CH₂—, —CH₂CH₂—, —NR₇—, -Q-N(R₇)—, —CHR₈—, —(CHR₈)₂—, —CHR₉—, —CR₉R₁₀—, —CO—, —O—, —OCH₂—, —OCHR₉—, or —OCR₉R₁₀—; wherein R₇ and R₈ are as previously defined; and Q is C₁₋₆ alkylenyl;
each R₉ and each R₁₀ is independently C₁₋₃ alkyl or OH; or
any R₉ and R₁₀, together with the atom to which they are attached, can form a 3 to 7 membered saturated ring that optionally contains one O, N or S atom;
X is CO₂R₁₁, COR₁₁, C(R₁₁)₂OH, CO₂R₅, C(O)NR₅R₆, NR₅R₆, QNR₅CO₂R₆, OH, CH₂OH, CN, SO₂NR₅R₆, P(O)(OR₅)(OR₆), cycloalkylalkyl, aryl, arylalkyl, heterocycloalkyl or heteroaryl, wherein:
said aryl, said arylalkyl, said heterocycloalkyl and said heteroaryl are independently each optionally substituted with up to three substituents selected from the group consisting of C₁₋₃ alkyl, C₁₋₃ alkoxy, halogen, H, OH, NO₂ and benzyl that is optionally substituted with up to five halogen atoms; wherein said C₁₋₃ alkyl and said C₁₋₃ alkoxy are each optionally substituted with from 1 to 7 fluorine atoms;
Q is C₁₋₆ alkylenyl;
R₁₁ is H or C₁₋₆ alkyl; and
R₅ and R₆ are as previously defined;
R₃ is C₁₋₈ alkyl, C₂₋₈ alkenyl, C₂₋₈ alkynyl, C₃₋₈ cycloalkyl, C₃₋₈ cycloalkenyl, phenyl, or ZA; wherein:
said phenyl is optionally substituted with C₁₋₃ alkyl;
Z is CH₂, CH₂CH₂, or CH₂O;
A is biphenyl, benzyl, naphthyl, pyridyl, 8-quinolyl, C₃₋₈ cycloalkyl or phenyl;
wherein said phenyl is optionally substituted with up to five independently selected R₁₈ groups; wherein
each said R₁₈ is independently selected from the group consisting of C₁₋₆ alkyl, C₁₋₃ alkoxy, halogen, OH, NO₂, CN, phenyl, pyrrol-1-yl, C(O)R₁₂, CO₂R₁₂, NR₁₂R₁₃, C(O)NR₁₂R₁₃ and S(O)ₙR₁₂; wherein said C₁₋₆ alkyl and said C₁₋₃ alkoxy are each optionally substituted with from 1 to 7 fluorine atoms; n is 0, 1 or 2; and R₁₂ and R₁₃ are each independently H or C₁₋₃ alkyl;
R₂₀ is H or C₁₋₃ alkyl; and
R₄ is H, halogen, methyl or methoxy;
provided that when the compound has the structure (Ia), then R₂ is phenyl or heteroaryl, each of which is substituted by YD, wherein YD is as previously defined; or a pharmaceutically acceptable salt thereof.

Additionally, compounds disclosed in co-owned, copending U.S. Patent Application Publication No. 2005/0131014 are useful in the therapeutic or pharmaceutical compositions disclosed herein. Compounds disclosed therein include those having formula (V): 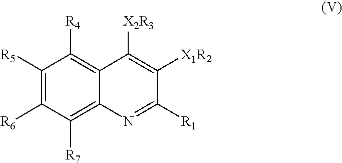
wherein:
R₁ is —H or C₁ to C₃ alkyl;
X₁ is a bond, C₁ to C₅ alkyl, —C(O)—, —C(═CR₈R₉)—, —O—, —S(O)ₜ—, —NR₈—, —CR₈R₉—, —CHR₂₃, —CR₈(OR₉)—, —C(OR₈)₂—, —CR₈(OC(O)R₉)—, —C═NOR₉—, —C(O)NR₈—, —CH₂O—, —CH2S—, —CH₂NR₈—, —OCH2—, —SCH2—, —NR₈CH₂—, or 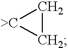
   R₂ is H, C₁ to C₆ alkyl, C₂ to C₆ alkenyl, C₂ to C₆ alkynyl, C₃ to C₆ cycloalkyl, —CH₂OH, C₇ to C₁₁ arylalkyl, phenyl, naphthyl, C₁ to C₃ perfluoroalkyl, CN, C(O)NH₂, CO₂R₁₂ or phenyl substituted independently by one or more of the groups independently selected from C₁ to C₃ alkyl, C₂ to C₄ alkenyl, C₂ to C₄ alkynyl, C₁ to C₃ alkoxy, C₁ to C₃ perfluoroalkyl, halogen, —NO₂, —NR₈R₉, —CN, —OH, and C₁ to C₃ alkyl substituted with 1 to 5 fluorines, or
R₂ is a heterocycle selected from the group consisting of pyridine, thiophene, benzisoxazole, benzothiophene, oxadiazole, pyrrole, pyrazole, imidazole and furan, each of which may be optionally substituted with one to three groups independently selected from C₁ to C₃ alkyl, C₁ to C₃ alkoxy, C₁ to C₃ perfluoroalkyl, halogen, —NO₂, —NR₈R₉, —CN, and C₁ to C₃ alkyl substituted with 1 to 5 fluorines;
X₂ is a bond or —CH₂—;
R₃ is phenyl, naphthyl, or phenyl or naphthyl substituted by one to four groups independently selected from C₁ to C₃ alkyl, hydroxy, phenyl, acyl, halogen, —NH₂, —CN, —NO₂, C₁ to C₃ alkoxy, C₁ to C₃ perfluoroalkyl, C₁ to C₃ alkyl substituted with 1 to 5 fluorines, NR₁₄R₁₅, —C(O)R₁₀, —C(O)NR₁₀R₁₁, —C(O)NR₁₁A, —C≡CR₈, —CH═CHR₈, —WA, —C≡CA, —CH═CHA, —WYA, —WYNR₁₁-A, —WYR₁₀, —WY(CH₂)ⱼA, —WCHR₁₁(CH₂)ⱼA, —W(CH₂)ⱼA, —W(CH₂)ⱼR₁₀, —CHR₁₁W(CH₂)ⱼR₁₀, —CHR₁₁W(CH₂)ⱼA, —CHR₁₁NR₁₂YA, —CHR₁₁NR₁₂YR₁₀, pyrrole, —W(CH₂)ⱼA(CH₂)ₖD(CH₂)ₚZ, —W(CR₁₈R₁₉)A(CH₂)ₖD(CH₂)ₚZ, —(CH₂)ⱼWA(CH₂)ₖD(CH₂)ₚZ, —CH═CHA(CH₂)ₖD(CH₂)ₚZ, —C≡CA(CH₂)ₖD(CH₂)ₚZ, —W(CH₂)ⱼC≡CA(CH₂)ₖD(CH₂)ₚZ, and —W(CH₂)ⱼZ, or
R₃ is a heterocycle selected from pyridine, pyrimidine, thiophene, furan, benzothiophene, indole, benzofuran, benzimidazole, benzothiazole, benzoxazole, and quinoline each of which may be optionally substituted with one to three groups independently selected from C₁ to C₃ alkyl, C₁ to C₃ alkoxy, hydroxy, phenyl, acyl, halogen, —NH₂, —CN, —NO₂, C₁ to C₃ perfluoroalkyl, C₁ to C₃ alkyl substituted with 1 to 5 fluorines, —C(O)R₁₀, —C(O)NR₁₀R₁₁, —C(O)NR₁₁A, —C≡CR₈, —CH═CHR₈, —WA, —C≡CA, —CH═CHA, —WYA, —WYR₁₀, —WY(CH₂)ⱼA, —W(CH₂)ⱼA, —W(CH₂)ⱼR₁₀, —CHR₁₁W(CH₂)ⱼR₁₀, —CHR₁₁W(CH₂)ⱼA, —CHR₁₁NR₁₂YA, —CHR₁₁NR₁₂YR₁₀, —WCHR₁₁(CH₂)ⱼA, —W(CH₂)ⱼA(CH₂)ₖD(CH₂)ₚZ, —W(CR₁₈R₁₉)A(CH₂)ₖD(CH₂)ₚZ, —(CH₂)ⱼWA(CH₂)ₖD(CH₂)ₚZ, —CH═CHA(CH₂)ₖD(CH₂)ₚZ, —C≡CA(CH₂)ₖD(CH₂)ₚZ, —W(CH₂)ⱼC≡CA(CH₂)ₖD(CH₂)ₚZ, and —W(CH₂)ⱼZ;
W is a bond, —O—, —S—, —S(O)—, —S(O)₂—, —NR₁₁—, or —N(COR₁₂)—;
Y is —CO—, —S(O)₂—, —CONR₁₃, —CONR₁₃CO—, —CONR₁₃SO₂—, —C(NCN)—, —CSNR₁₃, —C(NH)NR₁₃, or —C(O)O—;
j is 0 to 3;
k is 0 to 3;
t is 0 to 2;
D is a bond, —CH═CH—, —C═, —C(O)—, —C≡C—, phenyl, —O—, —NH—, —S—, —CHR₁₄—, —CR₁₄R₁₅—, —OCHR₁₄—, —OCR₁₄R₁₅—, or —CH(OH)CH(OH)—;
p is 0 to 6;
Z is —CO₂R₁₁, —CONR₁₀R₁₁, —C(═NR₁₀)NR₁₁R₁₂, —CONH₂NH₂, —CN, —CH₂OH, —NR₁₆R₁₇, phenyl, CONHCH(R₂₀)COR₁₂, phthalimide, pyrrolidine-2,5-dione, thiazolidine-2,4-dione, tetrazolyl, pyrrole, indole, oxazole, 2-thioxo-1,3-thiazolidin-4-one, C₁ to C₇ amines, C₃ to C₇ cyclic amines, or C₁ to C₃ alkyl substituted with one to two OH groups; wherein said pyrrole is optionally substituted with one or two substituents independently selected from the group consisting of —CO₂CH₃, —CO₂H, —COCH₃, —CONH₂ and —CN; wherein said C₁ to C₇ amines are optionally substituted with one to two substituents independently selected from the group consisting of —OH, halogen, —OCH₃, and —C≡CH; wherein said phenyl is optionally substituted with CO₂R₁₁, wherein said C₃ to C₇ cyclic amines are optionally substituted with one or two substituents independently selected from the group consisting of —OH —CH₂OH, —CH₂OCH₃, —CO₂CH₃, and —CONH₂, and wherein said oxazole is optionally substituted with CH₂CO₂R₁₁;
A is phenyl, naphthyl, tetrahydronaphthyl, indan, or biphenyl, each of which may be optionally substituted by one to four groups independently selected from halogen, C₁ to C₃ alkyl, C₂ to C₄ alkenyl, C₂ to C₄ alkynyl, acyl, hydroxy, halogen, —CN, —NO₂, —CO₂R₁₁, —CH₂CO₂R₁₁, phenyl, C₁ to C₃ perfluoroalkoxy, C₁ to C₃ perfluoroalkyl, —NR₁₀R₁₁, —CH₂NR₁₀R₁₁, —SR₁₁, C₁ to C₆ alkyl substituted with 1 to 5 fluorines, C₁ to C₃ alkyl substituted with 1 to 2 —OH groups, C₁ to C₆ alkoxy optionally substituted with 1 to 5 fluorines, or phenoxy optionally substituted with 1 to 2 CF₃ groups; or
A is a heterocycle selected from pyrrole, pyridine, pyridine-N-oxide, pyrimidine, pyrazole, thiophene, furan, quinoline, oxazole, thiazole, imidazole, isoxazole, indole, benzo[1,3]-dioxole, benzo[1,2,5]-oxadiazole, isochromen-1-one, benzothiophene, benzofuran, 2,3-dihydrobenzo[1,4]-dioxine, bithienyl, quinazolin-2,4-[1,3H]dione, and 3-H-isobenzofuran-1-one, each of which may be optionally substituted by one to three groups independently selected from halogen, C₁ to C₃ alkyl, acyl, hydroxy, —CN, —NO₂, C₁ to C₃ perfluoroalkyl, —NR₁₀R₁₁, —CH₂NR₁₀R₁₁, —SR₁₁, C₁ to C₆ alkyl substituted with 1 to 5 fluorines, and C₁ to C₃ alkoxy optionally substituted with 1 to 5 fluorines;
R₄, R₅, and R₆ are each, independently, —H or —F;
R₇ is hydrogen, C₁ to C₄ alkyl, C₁ to C₄ perfluoroalkyl, halogen, —NO₂ or —CN, phenyl or phenyl substituted with one or two group independently selected from halogen, C₁ to C₂ alkyl and OH;
   provided that if R₇ is hydrogen, then R₃ is selected from:
      (a) phenyl substituted by —W(CH₂)ⱼA(CH₂)ₖD(CH₂)ₚZ, —W(CR₁₈R₁₉)A(CH₂)ₖD(CH₂)ₚZ, —(CH₂)ⱼWA(CH₂)ₖD(CH₂)ₚZ, —CH═CHA(CH₂)ₖD(CH₂)ₚZ, —C≡CA(CH₂)ₖD(CH₂)ₚZ, or —W(CH₂)ⱼC≡CA(CH₂)ₖD(CH₂)ₚZ, wherein the phenyl moiety is further optionally substituted with one or two groups independently selected from C₁ to C₂ alkyl, C₁ to C₂ perfluoroalkyl, halogen, and CN; and
      (b) a heterocycle selected from pyridine, pyrimidine, thiophene, and furan, each of which is substituted by one of —W(CH₂)ⱼA(CH₂)ₖD(CH₂)ₚZ, —W(CR₁₈R₁₉)A(CH₂)ₖD(CH₂)ₚZ, —(CH₂)ⱼWA(CH₂)ₖD(CH₂)ₚZ, —CH═CHA(CH₂)ₖD(CH₂)ₚZ, —C≡CA(CH₂)ₖD(CH₂)ₚZ, or —W(CH₂)ⱼC≡CA(CH₂)ₖD(CH₂)ₚZ;
   further provided that if XI R₂ forms hydrogen, then R₃ is selected from:
      (a) phenyl substituted by —W(CH₂)ⱼA(CH₂)ₖD(CH₂)ₚZ, —W(CR₁₈R₁₉)A(CH₂)ₖD(CH₂)ₚZ, —(CH₂)ⱼWA(CH₂)ₖD(CH₂)ₚZ, —CH═CHA(CH₂)ₖD(CH₂)ₚZ, —C≡CA(CH₂)ₖD(CH₂)ₚZ, or —W(CH₂)ⱼC≡CA(CH₂)ₖD(CH₂)ₚZ, wherein the phenyl moiety is further optionally substituted with one or two groups independently selected from C₁ to C₂ alkyl, C₁ to C₂ perfluoroalkyl, halogen, and CN; and
      (b) a heterocycle selected from pyridine, pyrimidine, thiophene, and furan, each of which is substituted by one of —W(CH₂)ⱼA(CH₂)ₖD(CH₂)ₚZ, —W(CR₁₈R₁₉)A(CH₂)ₖD(CH₂)ₚZ, —(CH₂)ⱼWA(CH₂)ₖD(CH₂)ₚZ, —CH═CHA(CH₂)ₖD(CH₂)ₚZ, —C≡CA(CH₂)ₖD(CH₂)ₚZ, or —W(CH₂)ⱼC≡CA(CH₂)ₖD(CH₂)ₚZ;
   further provided that R₃ and R₇ cannot both be hydrogen;
each R₈ is independently —H, or C₁ to C₃ alkyl;
each R₉ is independently —H, or C₁ to C₃ alkyl;
each R₁₀ is independently —H, —OH, C₁ to C₃ alkoxy, C₁ to C₇ alkyl, C₃ to C₇ alkenyl, C₃ to C₇ alkynyl, C₃ to C₇ cycloalkyl, —CH₂CH₂OCH₃, 2-methyl-tetrahydro-furan, 2-methyl-tetrahydro-pyran, 4-methyl-piperidine, morpholine, pyrrolidine, or phenyl optionally substituted with one or two C₁ to C₃ alkoxy groups, wherein said C₁ to C₇ alkyl is optionally substituted with 1, 2 or 3 groups independently selected from C₁ to C₃ alkoxy, C₁ to C₃ thioalkoxy and CN;
each R₁₁ is independently —H, C₁ to C₃ alkyl or R₂₂;
   or R₁₀ and R₁₁, when attached to the same atom, together with said atom form:
   a 5 to 7 membered saturated ring, optionally substituted by 1 to 2 groups independently selected from C₁ to C₃ alkyl, OH and C₁-C₃ alkoxy, or a 5 to 7 membered ring containing 1 or 2 heteroatoms, optionally substituted by 1 to 2 groups independently selected from C₁ to C₃ alkyl, OH and C₁-C₃ alkoxy;
each R₁₂ is independently —H, or C₁ to C₃ alkyl;
each R₁₃ is independently —H, or C₁ to C₃ alkyl;
each R₁₄ and R₁₅ is, independently, C₁ to C₇ alkyl, C₃ to C₈ cycloalkyl, C₂ to C₇ alkenyl, C₂ to C₇ alkynyl, —OH, —F, C₇ to C₁₄ arylalkyl, where said arylalkyl is optionally substituted with 1 to 3 groups independently selected from NO₂, C₁ to C₆ alkyl, C₁ to C₃ perhaloalkyl, halogen, CH₂CO₂R₁₁, phenyl and C₁ to C₃ alkoxy, or R₁₄ and R₁₅ together with the atom to which they are attached can form a 3 to 7 membered saturated ring;
each R₁₆ and R₁₇ is, independently, hydrogen, C₁ to C₃ alkyl, C₁ to C₃ alkenyl, C₁ to C₃ alkynyl, phenyl, benzyl or C₃ to C₈ cycloalkyl, wherein said C₁ to C₃ alkyl is optionally substituted with one OH group, and wherein said benzyl is optionally substituted with 1 to 3 groups independently selected from C₁ to C₃ alkyl and C₁ to C₃ alkoxy; or
R₁₆ and R₁₇, together with the atom to which they are attached, can form a 3 to 8 membered heterocycle which is optionally substituted with one or two substituents independently selected from the group consisting of C₁ to C₃ alkyl, —OH, CH₂OH, —CH₂OCH₃, —CO₂CH₃, and —CONH₂;
each R₁₈ and R₁₉ is, independently, C₁ to C₃ alkyl;
each R₂₀ is independently H, phenyl, or the side chain of a naturally occurring alpha amino acid;
each R₂₂ is independently arylalkyl optionally substituted with CH₂CO₂H; and
each R₂₃ is phenyl;
or a pharmaceutically acceptable salt thereof.
   III. Methods of Treatment/Prevention

According to one modulatory method, LXR activity is stimulated in a cell by contacting the cell with an LXR modulator. Examples of such LXR modulators are described above in Section II. Other LXR modulators that can be used to stimulate the LXR activity can be identified using screening assays that select for such compounds, as described in detail herein (Section V).

1. Prophylactic Methods

In one aspect, the invention provides a method for preventing in a subject skin aging by administering to the subject an LXR modulator. Administration of a prophylactic LXR modulator can occur prior to the manifestation of skin aging symptoms, such that skin aging is prevented or, alternatively, delayed in its progression.

2. Therapeutic Methods

Another aspect of the invention pertains to methods of modulating LXR activity for the treatment of skin aging. Accordingly, in an exemplary embodiment, a modulatory method of the invention involves contacting a cell with an LXR modulator that induces TIMP1, ASAH1, SPTLC1, SMPD1, LASS2, TXNRD1, GPX3, GSR, CAT, ABCA1, ABCA2, ABCA12, ABCA13, ABCG1, and/or decorin expression and/or inhibits TNFα, MMP1, MMP3, and/or IL-8 expression. These modulatory methods can be performed in vitro (e.g., by culturing the cell with an LXR modulator) or, alternatively, in vivo (e.g., by administering an LXR modulator to a subject). As such, the present invention provides methods of treating a subject affected by skin aging that would benefit from induction of TIMP1, ASAH1, SPTLC1, SMPD1, LASS2, TXNRD1, GPX3, GSR, CAT, ABCA1, ABCA2, ABCA12, ABCA13, ABCG1, and/or decorin expression and/or inhibition of TNFα, MMP1, MMP3, and/or IL-8 expression.

IV. Administration of LXR Modulators

LXR modulators are administered to subjects in a biologically compatible form suitable for topical administration to treat or prevent skin aging. By “biologically compatible form suitable for topical administration” is meant a form of the LXR modulator to be administered in which any toxic effects are outweighed by the therapeutic effects of the modulator. The term “subject” is intended to include living organisms in which an immune response can be elicited, for example, mammals. Administration of LXR modulators as described herein can be in any pharmacological form including a therapeutically effective amount of an LXR modulator alone or in combination with a pharmaceutically acceptable carrier.

The therapeutic or pharmaceutical compositions of the present invention can be administered by any other suitable route known in the art including, for example, oral, intravenous, subcutaneous, intramuscular, or transdermal, or administration to cells in ex vivo treatment protocols. Administration can be either rapid as by injection or over a period of time as by slow infusion or administration of slow release formulation. For treating or preventing skin aging, administration of the therapeutic or pharmaceutical compositions of the present invention can be performed, for example, by topical administration.

Topical administration of an LXR modulator may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Chapter 67 of Remington's Pharmaceutical Sciences, 15th Edition (1975) published by Mack Publishing Company.

Dermal or skin patches are another method for transdermal delivery of the therapeutic or pharmaceutical compositions of the invention. Patches can provide an absorption enhancer such as DMSO to increase the absorption of the compounds. Patches can include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin. The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

A therapeutically effective amount of an LXR modulator may vary according to factors such as the skin aging state, age, sex, and weight of the individual, and the ability of the LXR modulator to elicit a desired response in the individual. Dosage regime may be adjusted to provide the optimum cosmetic, response. For example, several divided doses may be administered daily, or the dose may be proportionally reduced as indicated by the exigencies of the skin aging.

LXR modulators can also be linked or conjugated with agents that provide desirable pharmaceutical or pharmacodynamic properties. For example, LXR modulators can be stably linked to a polymer such as polyethylene glycol to obtain desirable properties of solubility, stability, half-life, and other pharmaceutically advantageous properties (see, e.g., Davis et al., Enzyme Eng. 4:169-73 (1978); Burnham N L, Am. J. Hosp. Pharm. 51:210-18 (1994)).

LXR modulators can be in a composition which aids in delivery into the cytosol of a cell. For example, an LXR modulator may be conjugated with a carrier moiety such as a liposome that is capable of delivering the modulator into the cytosol of a cell. Such methods are well known in the art (see, e.g., Amselem S et al., Chem. Phys. Lipids 64:219-37 (1993)).

LXR modulators can be employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the LXR modulator, use thereof in the cosmetic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous.

In one embodiment, the anti-skin aging compositions disclosed herein can further comprise a retinoic acid receptor (RAR) ligand. Useful RAR ligands include, for example, all-trans retinoic acid (tretinoin) and/or synthetic retinoic acid receptor ligands. Tretinoin is sold under such trademarks as Atragen®, Avita®, Renova®, Retin-A®, Vesanoid®, and Vitinoin®. Exemplary synthetic retinoic acid receptor ligands include tazarotene (Avage®; ethyl 6-[2-(4,4-dimethylthiochroman-6-yl)ethynyl]pyridine-3-carboxylate) and Differin® (adapalene; 6-[3-(1-adamantyl)-4-methoxyphenyl]-2-naphthoic acid; CD271).

Topical compositions can be prepared by combining the anti-skin aging composition with conventional pharmaceutically acceptable diluents and carriers commonly used in topical dry, liquid, cream, and aerosol formulations. Ointment and creams can, for example, be formulated with an aqueous or oily base with the addition of suitable thickening and/or gelling agents. An exemplary base is water. Thickening agents which can be used according to the nature of the base include aluminum stearate, cetostearyl alcohol, propylene glycol, polyethylene glycols, hydrogenated lanolin, and the like. Lotions can be formulated with an aqueous base and will, in general, also include one or more of the following: stabilizing agents, emulsifying agents, dispersing agents, suspending agents, thickening agents, coloring agents, perfumes, and the like. Powders can be formed with the aid of any suitable powder base, for example, talc, lactose, starch, and the like. Drops can be formulated with an aqueous base or non-aqueous base, and can also include one or more dispersing agents, suspending agents, solubilizing agents, and the like.

In one embodiment, the topical composition may, for example, take the form of hydrogel based on polyacrylic acid or polyacrylamide; as an ointment, for example with polyethyleneglycol (PEG) as the carrier, like the standard ointment DAB 8 (50% PEG 300, 50% PEG 1500); or as an emulsion, especially a microemulsion based on water-in-oil or oil-in-water, optionally with added liposomes. Suitable permeation accelerators (entraining agents) include sulphoxide derivatives such as dimethylsulphoxide (DMSO) or decylmethylsulphoxide (decyl-MSO) and transcutol (diethyleneglycolmonoethylether) or cyclodextrin; as well as pyrrolidones, for example 2-pyrrolidone, N-methyl-2-pyrrolidone, 2-pyrrolidone-5-carboxylic acid, or the biodegradable N-(2-hydroxyethyl)-2-pyrrolidone and the fatty acid esters thereof; urea derivatives such as dodecylurea, 1,3-didodecylurea, and 1,3-diphenylurea; terpenes, for example D-limonene, menthone, a-terpinol, carvol, limonene oxide, or 1,8-cineol.

Ointments, pastes, creams and gels also can contain excipients, such as starch, tragacanth, cellulose derivatives, polyethylene glycols, silicones, bentonites, silicic acid, and talc, or mixtures thereof. Powders and sprays also can contain excipients such as lactose, talc, silicic acid, aluminum hydroxide, calcium silicates and polyamide powder, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such a chlorofluorohydrocarbons and volatile unsubstituted hydrocarbons, such as butane and propane.

The carrier can also contain other pharmaceutically-acceptable excipients for modifying or maintaining the pH, osmolarity, viscosity, clarity, color, sterility, stability, rate of dissolution, or odor of the formulation. The anti-skin aging compositions can also further comprise antioxidants, sun screens, natural retinoids (e.g., retinol), and other additives commonly found in skin treatment compositions.

Dose administration can be repeated depending upon the pharmacokinetic parameters of the dosage formulation and the route of administration used.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the LXR modulator and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the LXR modulator activities disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such LXR modulators can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. LXR modulators that exhibit large therapeutic indices are preferred. While LXR modulators that exhibit toxic side effects may be used, care should be taken to design a delivery system that targets such modulators to the site of affected tissue in order to minimize potential damage to uninfected cells and, thereby, reduce side effects.

The data obtained from the cell culture assays and animal studies can be used in formulating a range of dosage for use in humans. The dosage of such LXR modulators lies preferably within a range of circulating concentrations that include the ED₅₀ with little or no toxicity. The dosage may vary within this range depending upon the dosage form employed and the route of administration utilized. For any LXR modulator used in a method of the invention, the therapeutically effective dose can be estimated initially from cell culture assays. A dose may be formulated in animal models to achieve a circulating plasma concentration range that includes the IC₅₀ (i.e., the concentration of LXR modulator that achieves a half-maximal inhibition of symptoms) as determined in cell culture. Such information can be used to more accurately determine useful doses in humans. Levels in plasma may be measured, for example, by high performance liquid chromatography.

Monitoring the influence of LXR modulators on the induction of TIMP1, ASAH1, SPTLC1, SMPD1, LASS2, TXNRD1, GPX3, GSR, CAT, ABCA1, ABCA2, ABCA12, ABCA13, ABCG1, and/or decorin expression and/or inhibition of TNFα, MMP1, MMP3, and/or IL-8 expression can be applied in clinical trials. For example, the effectiveness of an LXR modulator can be monitored in clinical trials of subjects exhibiting increased TIMP1, ASAH1, SPTLC1, SMPD1, LASS2, TXNRD1, GPX3, GSR, CAT, ABCA1, ABCA2, ABCA12, ABCA13, ABCG1, and/or decorin expression and/or decreased TNFα, MMP1, MMP3, and/or IL-8 expression. In such clinical trials, the expression of TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8 can be used as a “read out” or markers of the different skin aging phenotypes.

Thus, to study the effect of LXR modulators on skin aging, for example, in a clinical trial, cells can be isolated and RNA prepared and analyzed for the levels of expression of TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8. The levels of gene expression (i.e., a gene expression pattern) can be quantified, for example, by Northern blot analysis or RT-PCR, by measuring the amount of protein produced, or by measuring the levels of activity of TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8, all by methods well known to those of ordinary skill in the art. In this way, the gene expression pattern can serve as a marker, indicative of the physiological response of the cells to the LXR modulator. Accordingly, this response state may be determined before, and at various points during, treatment of the individual with the LXR modulator.

The present invention also provides a method for monitoring the effectiveness of treatment of a subject with an LXR modulator comprising the steps of (i) obtaining a pre-administration sample from a subject prior to administration of the LXR modulator; (ii) detecting the level of expression of TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8; (iii) obtaining one or more post-administration samples from the subject; (iv) detecting the level of expression of TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8 in the post-administration samples; (v) comparing the level of expression of TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8 in the pre-administration sample with the TIMP1, ABCA12, decorin, TNFα, MMP1, MMP3, and/or IL-8 expression in the post administration sample or samples; and (vi) altering the administration of the LXR modulator to the subject accordingly. For example, increased administration of the LXR modulator may be desirable to increase TIMP1, ASAH1, SPTLC1, SMPD1, LASS2, TXNRD1, GPX3, GSR, CAT, ABCA1, ABCA2, ABCA12, ABCA13, ABCG1, and/or decorin expression to higher levels than detected and/or reduce TNFα, MMP1, MMP3, and/or IL-8 expression to lower levels than detected, that is, to increase the effectiveness of the LXR modulator. Alternatively, decreased administration of the LXR modulator may be desirable to decrease TIMP1, ASAH1, SPTLC1, SMPD1, LASS2, TXNRD1, GPX3, GSR, CAT, ABCA1, ABCA2, ABCA12, ABCA13, ABCG1, and/or decorin expression to lower levels than detected or activity and/or to increase TNFα, MMP1, MMP3, and/or IL-8 expression to higher levels than detected, that is, to decrease the effectiveness of the LXR modulator. According to such an embodiment, TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8 expression may be used as an indicator of the effectiveness of an LXR modulator, even in the absence of an observable phenotypic response.

Furthermore, in the treatment of skin aging, compositions containing LXR modulators can be administered exogenously, and it would likely be desirable to achieve certain target levels of LXR modulator in sera, in any desired tissue compartment, and/or in the affected tissue. It would, therefore, be advantageous to be able to monitor the levels of LXR modulator in a patient or in a biological sample including a tissue biopsy sample obtained from a patient and, in some cases, also monitoring the levels of TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8 expression. Accordingly, the present invention also provides methods for detecting the presence of LXR modulator in a sample from a patient.

V. Screening Assays

In one embodiment, expression levels of cytokines and metalloproteases described herein can be used to facilitate design and/or identification of compounds that treat skin aging through an LXR-based mechanism. Accordingly, the invention provides methods (also referred to herein as “screening assays”) for identifying modulators, i.e., LXR modulators, that have a stimulatory or inhibitory effect on, for example, TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8 expression. Compounds thus identified can be used as anti-skin aging compounds as described elsewhere herein.

Test compounds can be obtained, for example, using any of the numerous approaches in combinatorial library methods known in the art, including spatially addressable parallel solid phase or solution phase libraries; synthetic library methods requiring deconvolution; the ‘one-bead one-compound’ library method; and synthetic library methods using affinity chromatography selection.

Examples of methods for the synthesis of molecular libraries can be found in, for example: DeWitt S H et al., Proc. Natl. Acad. Sci. U.S.A. 90:6909-13 (1993); Erb E et al., Proc. Natl. Acad. Sci. USA 91:11422-26 (1994); Zuckermann R N et al., J. Med. Chem. 37:2678-85 (1994); Cho C Y et al., Science 261:1303-05 (1993); Carrell et al., Angew. Chem. Int. Ed. Engl. 33:2059 (1994); Carrell et al., Angew. Chem. Int. Ed. Engl. 33:2061 (1994); Gallop M A et al., J. Med. Chem. 37:1233-51 (1994).

Libraries of compounds may be presented in solution (e.g., Houghten R A et al., Biotechniques. 13:412-21 (1992)), or on beads (Houghten R A et al., Nature 354:82-84 (1991)), chips (Fodor S A et al., Nature 364:555-56 (1993)), bacteria (U.S. Pat. No. 5,223,409), spores (U.S. Pat. No. 5,223,409), plasmids (Cull M G et al., Proc. Natl. Acad. Sci. USA 89:1865-69 (1992)) or on phage (Scott J K & Smith G P, Science 249:386-90 (1990); Devlin J J et al., Science 249:404-06 (1990); Cwirla S E et al., Proc. Natl. Acad. Sci. 87:6378-82 (1990); Felici F et al., J. Mol. Biol. 222:301-10 (1991); U.S. Pat. No. 5,223,409.).

An exemplary screening assay is a cell-based assay in which a cell that expresses LXR is contacted with a test compound, and the ability of the test compound to modulate TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8 expression through an LXR-based mechanism. Determining the ability of the test compound to modulate TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8 expression can be accomplished by monitoring, for example, DNA, mRNA, or protein levels, or by measuring the levels of activity of TIMP1, ASAH1, SPTLC1, SMPD1, LASS2, TXNRD1, GPX3, GSR, CAT, ABCA1, ABCA2, ABCA12, ABCA13, ABCG1, decorin, TNFα, MMP1, MMP3, and/or IL-8, all by methods well known to those of ordinary skill in the art. The cell, for example, can be of mammalian origin, e.g., human.

Novel modulators identified by the above-described screening assays can be used for treatments as described herein.

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modification of the invention to adapt it to various uses and conditions.

RNA Extraction

Add QIAzol® Lysis Reagent (QIAGEN Cat Number 79306) to the cells. Scrape the cells and place into a Falcon Polypropylene tube. Let stand at room temperature for 5 minutes. Add 1 ml of cells to microfuge tubes. Add 200 μl of chloroform, vortex, let stand for 5 minutes. Centrifuge at 4° C. for 15 minutes at 14,000 RPM. Add an equal volume of 70% ETOH (diluted with DEPC water). Add 600 μl to the RNeasy® column from the RNeasy® Mini Kit (QIAGEN Cat. Number 74106) centrifuge at 14,000 RPM at room temperature for 1 minute, discard flow-through. Add remainder of sample to the column, centrifuge, discard flow-through. Add 350 μl of RW1 buffer from the RNeasy® Mini Kit to the column, centrifuge at room temperature for 1 minute, discard flow-through. DNase column with RNase-Free DNase Set (QIAGEN cat. Number 79254) by making DNase I stock solution, add 550 μl of water to the DNase, add 10 μl of DNase to 70 μl of BufferRDD for each sample, mix, add 80 μl to the column, let stand for 15 minutes. Add 350 μl of RW1 buffer to column, centrifuge for 1 minute, discard flow-through. Add 500 μl RPE buffer to column, centrifuge for 1 minute, discard flow-through. Add 500 μl RPE buffer to column, centrifuge for 1 minute, discard flow-through. Put column into a clean 2.0 ml microfuge tube, centrifuge for 2 minutes. Put column into a microfuge tube, add 50 μl of water, allow column to stand for 2 minutes, centrifuge for 1 minute.

Quantitative PCR

TaqMan technology was used for quantitative PCR for the evaluation of MMP, TNFα, TIMP, IL-8, ASAH1, SPTLC1, SMPD1, LASS2, TXNRD1, GPX3, GSR, CAT, ABCA1, ABCA2, ABCA12, ABCA13, ABCG1, decorin, and LXRα/β gene expression in keratinocytes and fibroblasts.

Conditions for use of TaqMan Reverse Transcriptase Reagents (Applied Biosystems Cat. Number N808-0234): 10× RT buffer: 10 μl, MgCl₂solution: 22 μl, DNTP mix: 20 μl, Random Hexamers: 5 μl, Multi Scribe RT: 2.5 μl, RNase Inhibitor: 2.5 μl, 2 μg RNA. Thermocycler: 25° C.-10 minutes, 48° C.-30 minutes, 95° C.-5 minutes.

Setup TaqMan with QuantiTect Multiplex PCR Kit (QIAGEN cat. Number 204543): 2× master mix: 25 μl; Single Tube Assay: 2.5 μl; Applied Biosystems Primers Probe set (part number 4308329)—18S forward primer: 0.25 μl, 18S reverse primer: 0.25 μl, 18S probe: 0.25 μl; water to 50 μl; 5 μl cDNA. Thermocycler: 50° C. -2 minutes, 95° C.-10 minutes, 95° C.-15 seconds, 60° C.-1 minute.

### Example 1

Clonetics® Normal Human Epidermal Keratinocytes (NHEKs) were obtained from Cambrex Bio Science, Inc. The proliferating T-25 (C2503TA25) pooled, neonatal keratinocytes were expanded in Clonetics® KGM-2 serum-free medium (CC-3107) and subcultured as needed using the recommended Clonetics® ReagentPack™ (CC-5034). Due to a light-sensitive component in the medium, all manipulations were done in low light.

For experiments, 1.6 million NHEK cells were plated in growth medium on 100 mm dishes and allowed to grow to ˜75% confluence. On the day of treatment, the dishes were rinsed once with KGM-2 minus hydrocortisone; then, vehicle (0.1% DMSO) or 1 μM WAY-205014 (Tularik 0901317), an LXR agonist, was added for 6 h in hydrocortisone-deficient KGM-2. After 6 h, the treatment medium was temporarily removed, the dishes washed with Dulbecco's Phosphate Buffered Saline, and then half of the treatments were exposed to 8 J/m² ultraviolet light using a Stratagene UV Stratalinker® 2400. Treatments were replaced and 18 h later the samples were harvested for RNA processing using TRIzol®D Reagent (Invitrogen).

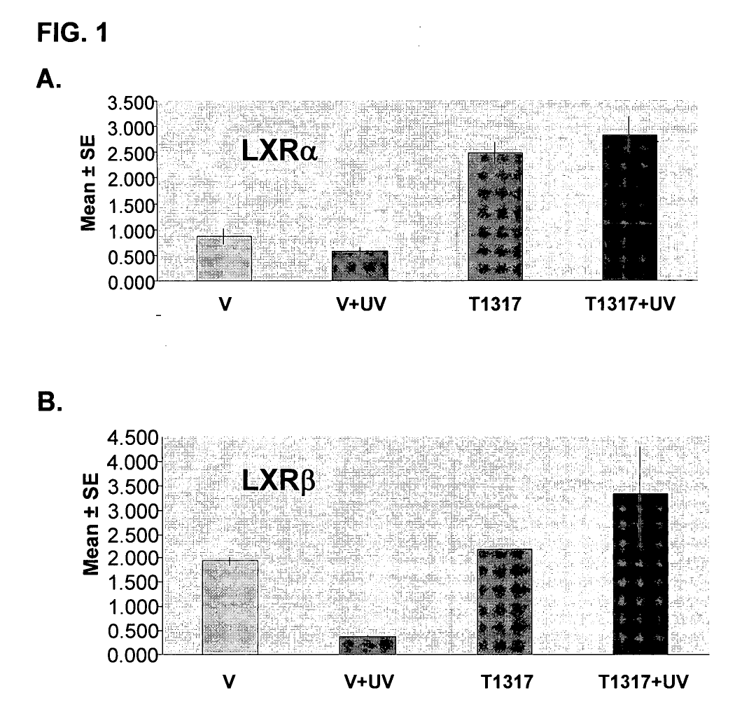

RNA was extracted as described above. FIG. 1A shows that the UV irradiation of NHEKs slightly reduced the expression of LXRα. Treatment of keratinocytes with the LXR modulator (1 μM) induced the expression of LXRα in both UV-unexposed and UV-exposed keratinocytes. FIG. 1B shows that the UV treatment of NHEKs resulted in a dramatic down-regulation of LXRβ expression, and this UV-mediated inhibition of LXRβ expression was reversed by treatment with the LXR modulator. Therefore, an LXR modulator induced the expression of both of its receptors in UV-exposed keratinocytes. These results further indicate that LXR modulators may help the UV-exposed keratinocytes/skin to be more responsive to its effects.

### Example 2

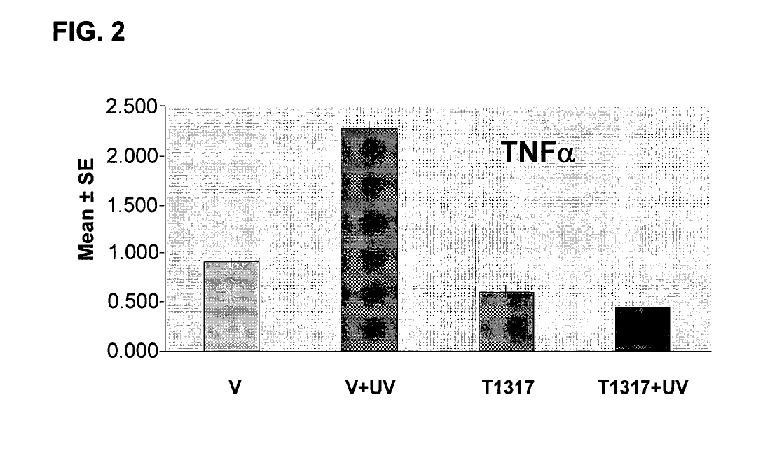

NHEK cells were treated and RNA extracted as described in Example 1. FIG. 2 shows that UV exposure of keratinocytes resulted in induction of TNFα expression. Further, the LXR modulator T1317 reduced both the basal expression of TNFα in UV-unexposed as well as the UV-induced expression of TNFα in keratinocytes. The reduced expression of UV-induced TNFα expression is expected to result in less activation of dermal fibroblasts, resulting in less production of metalloproteases that degrade the dermal matrix.

### Example 3

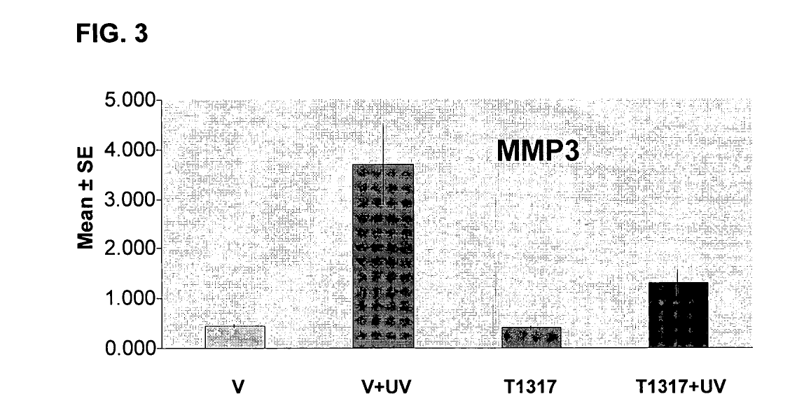

NHEK cells were treated and RNA extracted as described in Example 1. FIG. 3 shows that UV exposure of keratinocytes resulted in induction of MMP3 expression. Treatment of keratinocytes with the LXR modulator (T1317) resulted in inhibition of UV-induced MMP-3 expression in keratinocytes. The reduced expression of UV-induced MMP-3 expression is expected to result in reduced degradation of the dermal matrix.

### Example 4

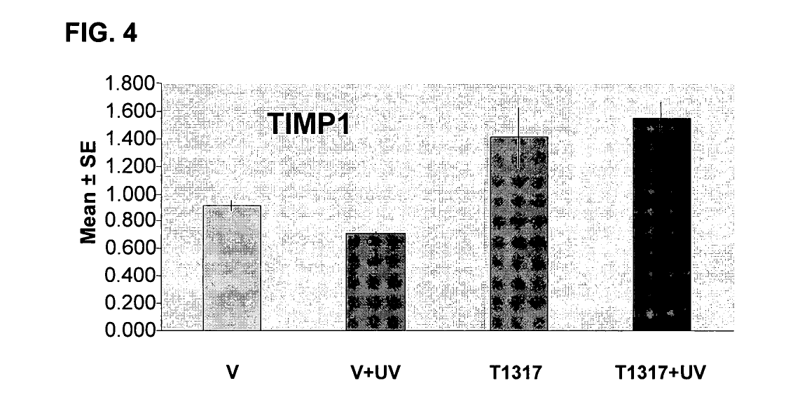

NHEK cells were treated and RNA extracted as described in Example 1. FIG. 4 shows that UV exposure of keratinocytes resulted in slight reduction of the basal level expression of TIMP1 expression. Interestingly, the LXR modulator T1317 induced TIMP1 expression in both UV-unexposed as well as UV-exposed keratinocytes. The induction of TIMP1 expression is expected to neutralize the metalloprotease activities, resulting in the protection of dermal matrix from the action of MMPs.

### Example 5

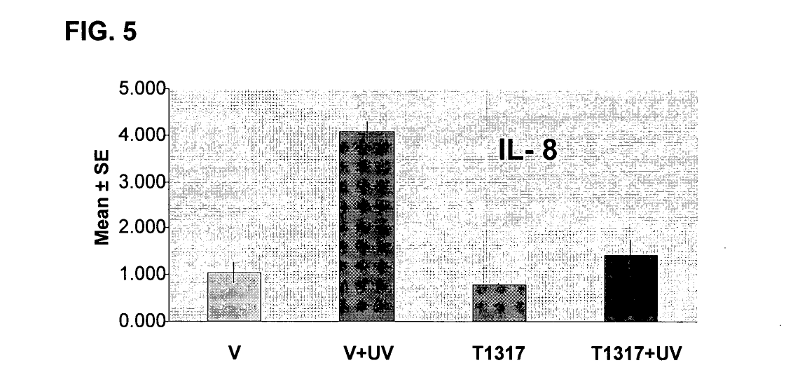

NHEK cells were treated and RNA extracted as described in Example 1. FIG. 5 shows that UV exposure of keratinocytes resulted in induction of IL-8 expression. Further, the LXR modulator T1317 reduced the UV-induced expression of IL-8 in keratinocytes. Because IL-8 is a chemotactic molecule, reduced expression of UV-induced IL-8 expression is expected to result in less recruitment of activated neutrophils into the dermis. Active neutrophils are also a source of MMPs and elastase that degrade the dermal matrix in photoaging.

### Example 6

Photoaged or photodamaged skin shows defective epidermal barrier function. ABCA12 is a lipid transporter that is essential for the maintenance and development of the epidermal barrier function of the skin.

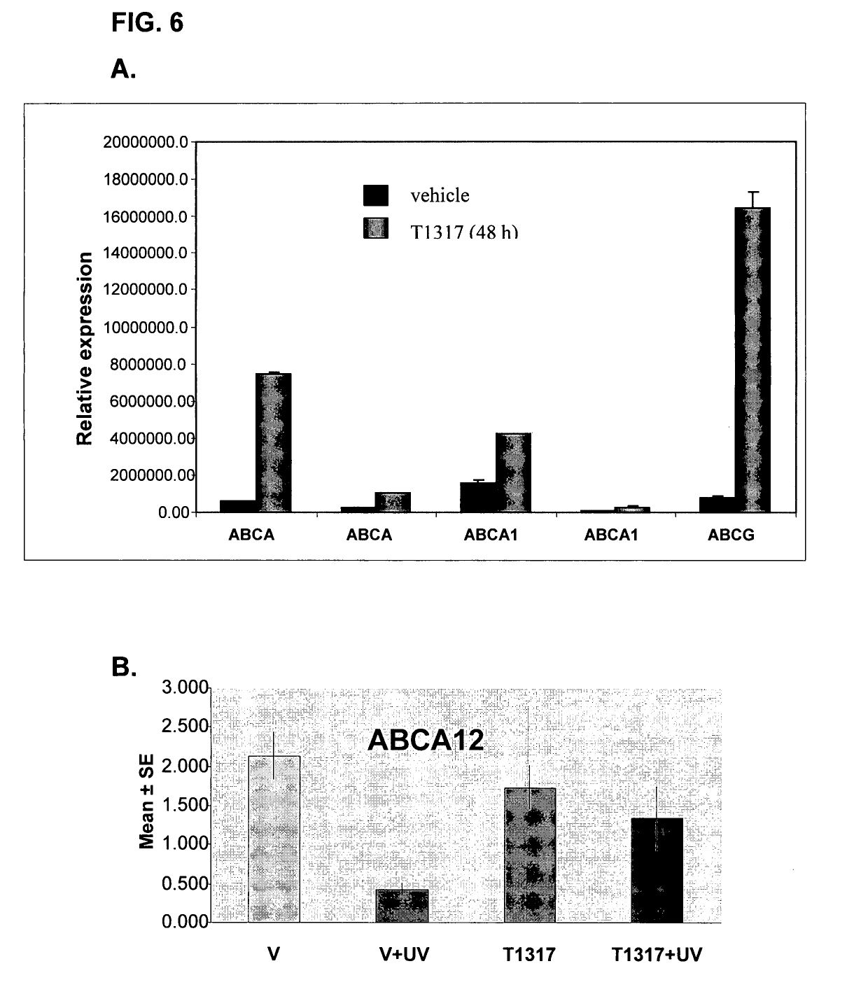

NHEK cells were treated and RNA extracted as described in Example 1. FIG. 6A shows that T1317 treatment of NHEKs resulted in the induction of ABCA1, ABCA2, ABCA12, ABCA13, and ABCG1 expression. Therefore, LXR ligands may induce the synthesis of lipids and their loading into epidermal lamellar bodies by inducing the expression of lipid binding proteins and ABC transporter family members required for cholesterol and lipid efflux These gene regulations also indicate that the LXR ligands may exhibit potent anti-xerosis therapeutic effect, thus alleviating one of the major symptoms of aged skin that leads to deterioration of epidermal barrier function and responsible for initiating other serious cutaneous conditions. NHEK cells were treated and RNA extracted as described in Example 1. Applicants observed a dramatic down-regulation of ABCA12 expression in UV-exposed keratinocytes (FIG. 6B). This UV-induced inhibition of ABCA12 expression was reversed by treatment with the LXR modulator T1317 (FIG. 6B). Increased ABCA12 expression by the LXR modulator may result in normalization of epidermal barrier function in the photoaged skin. Improved epidermal barrier function is expected to reduce skin dryness, a hallmark of photodamaged/photoaged skin.

### Example 7

Photoaged and chronologically aged skin shows decreased levels of collagen. Collagen is a component of the extracellular matrix that is required for imparting rigidity to cellular as well as dermal matrix structures. Collagen molecules are arranged in the form of collagen fibrils that is required for the normal architecture of the skin. This fibrillar architecture of the collagen is degraded in aged/wrinkled skin. Therefore, restoration of the collagen fibrillar structure is also expected to result in therapeutic improvement of the photodamaged/photoaged skin.

Decorin is an extracellular matrix component that associates with collagen I. Further, decorin-collagen interaction is required for collagen fibril formation. In other words, decorin is a critical regulator of collagen 1 fibrillar-genesis. Therefore, increased decorin expression in UV-exposed photodamaged skin is expected to induce the generation of collagen fibrils, a process that may improve skin laxity and wrinkles.

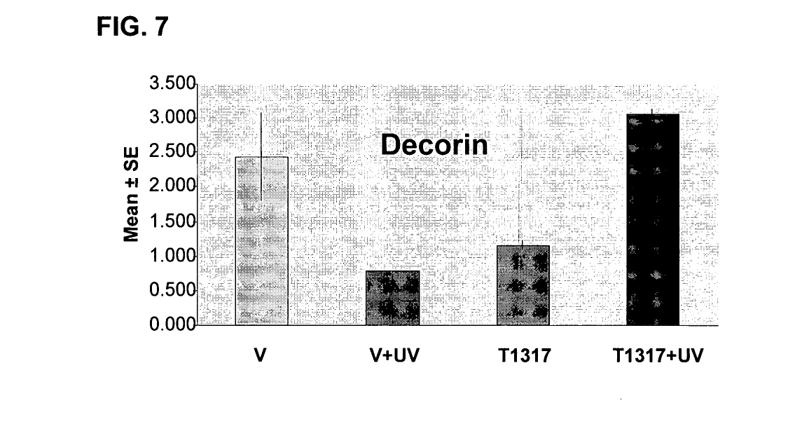

NHEK cells were treated and RNA extracted as described in Example 1. FIG. 7 shows that UV exposure of NHEKs resulted in a dramatic inhibition of decorin expression. The UVB-mediated inhibition of decorin expression was reversed by treatment with the LXR modulator. Therefore, LXR modulator normalized decorin expression in UV-exposed keratinocytes. The induction of decorin expression is expected to result in increased extracellular matrix formation.

### Example 8

The BJ cell line (ATCC # CRL-2522) was obtained from ATCC. It is a normal human fibroblast cell line originally derived from foreskin, demonstrating extended lifespan in culture of 80-90 population doublings. The cells were maintained in Eagle's Minimal Essential medium with Earle's BSS (EMEM) supplemented with penicillin-streptomycin, 1.0 mM sodium pyruvate, 0.1 mM non-essential amino acids, 2 mM GlutaMAX-1™ and 10% HyClone fetal bovine serum (FBS). With the exception of serum, all reagents were obtained from Invitrogen. The cells were subcultured with 0.05% trypsin-EDTA twice a week and maintained in a humidified incubator at 37° C. and 5% CO₂.

For experiments, 5 million BJ cells were plated in 150 mm dishes in growth medium. The following day, the phenol red-containing growth medium was removed and plates were rinsed once with phenol red-free EMEM without serum. Experimental medium was phenol red-free EMEM supplemented as above with the addition of 5% Lipoprotein Deficient Serum (Sigma S-5394) instead of HyClone FBS. DMSO vehicle (0.1%) or 1 μM WAY-205014 (Tularik 0701317), an LXR agonist, was added to the dishes for 6 h; at which time 5 ng/ml rhTNFα (R&D 210-TA) was added to half of the treatments. Samples were harvested with TRIzol® 18 h later and processed.

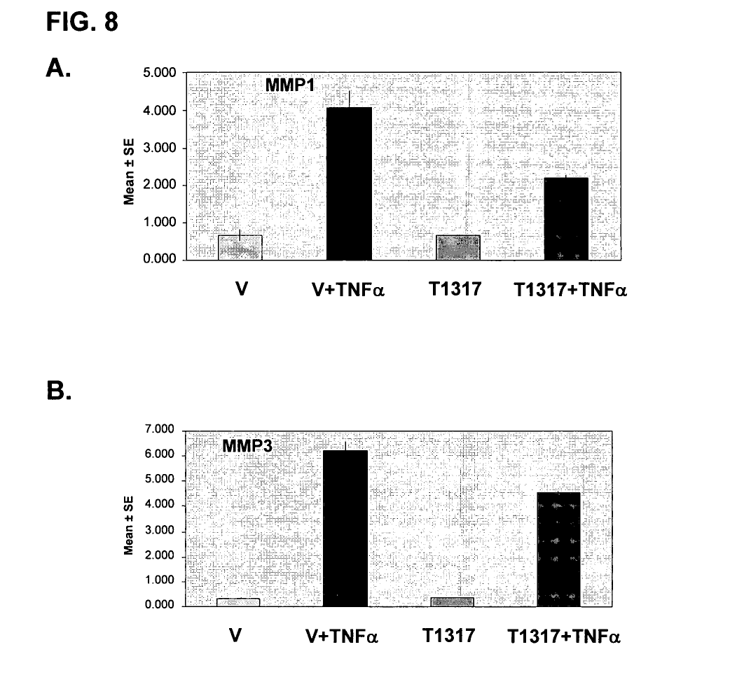

RNA was extracted as described above. FIG. 8A shows that TNFα treatment of BJ human fibroblasts resulted in the induction of MMP1 expression. Treatment of human fibroblasts with the LXR modulator (T1317) resulted in inhibition of TNFα-induced MMP1 expression. The reduced expression of TNFα-induced MMP1 expression is expected to result in reduced degradation of the dermal matrix because MMP1 is the major destroyer of the dermal matrix collagen.

### Example 9

BJ cells were treated and RNA extracted as described in Example 8. FIG. 8B shows that TNFα treatment of BJ human fibroblasts resulted in induction of MMP3 expression. Treatment of human fibroblasts with the LXR modulator (T1317) resulted in inhibition of TNFα-induced MMP-3 expression. The reduced expression of fibroblast TNFα-induced MMP-3 expression is expected to result in reduced degradation of the dermal matrix.

### Example 10

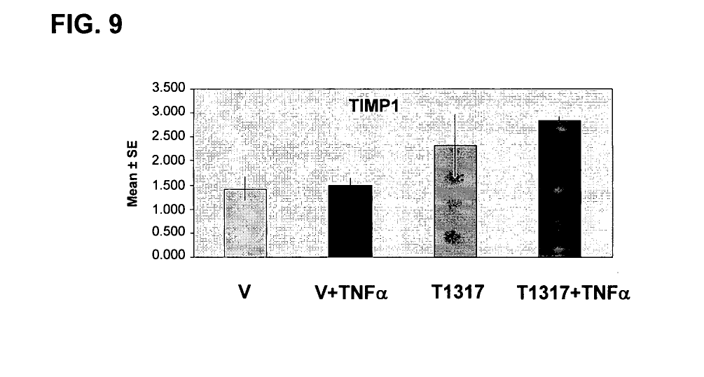

BJ cells were treated and RNA extracted as described in Example 8. FIG. 9 shows that unlike keratinocytes, TNFα exposure of human BJ fibroblasts did not result in reduction of the basal level expression of TIMP1 expression. Interestingly, the LXR modulator induced TIMP1 expression in both TNFα-unexposed as well as TNFα-exposed fibroblasts. The induction of TIMP1 expression is expected to neutralize the metalloprotease activities, resulting in the protection of dermal matrix from the action of MMPs.

### Example 11

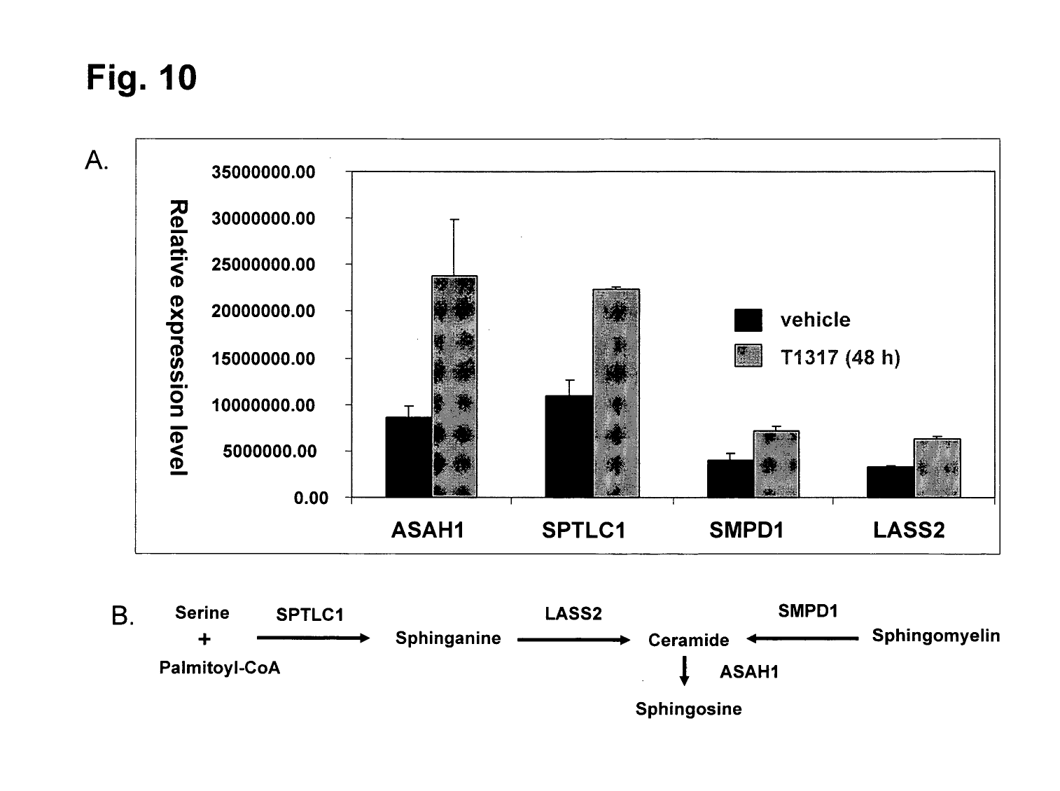

NHEK cells were treated and RNA extracted as described in Example 1. FIG. 10A shows that T1317 treatment of NHEKs resulted in induction of ASAH1, SPTLC1, SMPD1, and LASS2 expression. Ceramide is one of the major lipids in differentiated keratinocytes and it plays a pivotal role in skin barrier function. A comparison of chronologically aged and young skin revealed a decrease in ceramide content with age. The decline in ceramide content may result from reduced keratinocyte differentiation as well as because of reduced ceramide synthase and sphingomyelin (SM) phosphodiesterase activities in chronological aging. Serine palmitoyltransferase (SPTLC1) catalyzes the formation of sphinganine from serine and palmitoyl-CoA. Ceramide synthase (LASS2) converts sphinganine into ceramide. SM phosphodiesterase (SMPD) also produces ceramide from SM, and acid ceramidase (ASAH1) produces lipid second messenger sphingosine from ceramide. Here, Applicants demonstrated that the LXR lignad induced the expression of enzymes involved in ceramide and lipid second messenger sphingolipids biosynthetic pathway. Since ceramides and other sphingolipids are involved in keratinocyte proliferation, differentiation and desquamation, an increase in the expression of enzymes involved in the synthesis of sphingolipids may help in these processes and alleviate the epidermal problems (dry skin, decreased keratinocyte proliferation and differentiation, fine scales) that stem from decreased sphingolipid production.

### Example 12

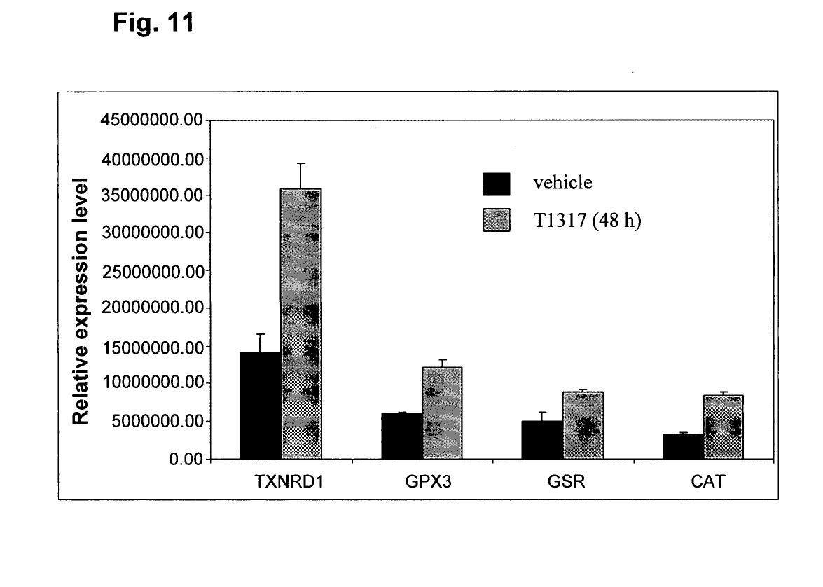

NHEK cells were treated and RNA extracted as described in Example 1. FIG. 11 shows that T1317 treatment of NHEKs resulted in induction of TXNRD1, GPX3, GSR, and CAT expression. UV-mediated cumulative oxidative damage in both epidermis and dermis due to accumulation of free radicals throughout life in all likelihood also promotes cellular aging. Free radicals or reactive oxygen species cause damage to lipids, protein and DNA, and cause cells to enter a senescent-like stage. There are many reports describing the reduction of antioxidant enzymes in skin with age, including superoxide dismutase, catalase and glutathione peroxidase. Therefore, Applicants examined the effect of the LXR ligand on the expression of enzymes involved in antioxidant activities in keratinocytes. Treatment of NHEKs with the synthetic LXR ligand induced the expression of anti-oxidant enzymes, glutathione peroxidase (GPX3), thioredoxin reductase, glutathione reductase and catalase. Therefore, LXR ligands may increase the free-radical fighting defense system of the body, which may reduce the insult of hydrogen peroxide and free-radicals on skin cell proteins, lipids and DNA.

What is claimed is:

1. An anti-skin aging composition comprising a therapeutically effective amount of an LXR modulator.

2. The anti-skin aging composition of claim 1, wherein the LXR modulator is a natural oxysterol, a synthetic oxysterol, a synthetic nonoxysterol, or a natural nonoxysterol.

3. The anti-skin aging composition of claim 1, wherein the LXR modulator is 20(S) hydroxycholesterol, 22(R) hydroxycholesterol, 24(S) hydroxycholesterol, 25-hydroxycholesterol, 24(S), 25 epoxycholesterol, 27-hydroxycholesterol, N,N-dimethyl-3β-hydroxycholenamide, N-(2,2,2-trifluoroethyl)-N-{4-[2,2,2-trifluoro-1-hydroxy-1-(trifluoromethyl)ethyl]phenyl}benzene sulfonamide, [3-(3-(2-chloro-trifluoromethylbenzyl-2,2-diphenylethylamino)propoxy)phenylacetic acid], N-methyl-N-[4-(2,2,2-trifluoro-1-hydroxy-1-trifluoromethyl-1-ethyl)-phenyl]-benzenesulfonamide, 4,5-dihydro-1-(3-(3-trifluoromethyl-7-propyl-benzisoxazol-6-yloxy)propyl)-2,6-pyrimidinedione, 3-chloro4-(3-(7-propyl-3-trifluoromethyl-6-(4,5)-isoxazolyl)propylthio)-phenyl acetic acid, acetyl-podocarpic dimer, paxilline, desmosterol, or stigmasterol.

4. The anti-skin aging composition of claim 3, wherein the LXR modulator is N-(2,2,2-trifluoroethyl)-N-{4-[2,2,2-trifluoro-1-hydroxy-1-(trifluoromethyl)ethyl]phenyl}benzene sulfonamide.

5. The anti-skin aging composition of claim 1 which is a topical anti-skin aging composition.

6. The anti-skin aging composition of claim 5, which is an anti-wrinkle cream.

7. The anti-skin aging composition of claim 1, wherein the therapeutically effective amount of LXR modulator induces expression of LXRα, LXRβ, or a combination thereof.

8. The anti-skin aging composition of claim 1, wherein the LXR modulator induces TIMP1 expression, induces ASAH1 expression, induces SPTLC1 expression, induces SMPD1 expression, induces LASS2 expression, induces TXNRD1 expression, induces GPX3 expression, induces GSR expression, induces CAT expression, induces ABCA1 expression, induces ABCA2 expression, induces ABCA12 expression, induces ABCA13 expression, induces ABCG1 expression, induces decorin expression, inhibits TNFα expression, inhibits MMP1 expression, inhibits MMP3 expression, inhibits IL-8 expression, or a combination thereof.

9. The anti-skin aging composition of claim 1, further comprising an RAR ligand.

10. The anti-skin aging composition of claim 9, wherein the RAR ligand is all-trans retinoic acid, ethyl 6-[2-(4,4-dimethylthiochroman-6-yl)ethynyl]pyridine-3-carboxylate, 6-[3-(1-adamantyl)-4-methoxyphenyl]-2-naphthoic acid, or a combination thereof.

11. The anti-skin aging composition of claim 1, further comprising antioxidant, sun screen, natural retinoid, or a combination thereof.

12. The anti-skin aging composition of claim 11, wherein the natural retinoid is retinol.

13. A method for the treatment of skin aging comprising administering to a mammal in need thereof a therapeutically effective amount of an LXR modulator.

14. The method of claim 13, wherein the mammal is a human.

15. The method of claim 13, wherein the skin aging is derived from chronological aging, photoaging, steroid-induced skin thinning, or a combination thereof.

16. The method of claim 15, wherein the chronological aging causes deepened expression lines, reduction of skin thickness, inelasticity, unblemished smooth surface, or a combination thereof.

17. The method of claim 15, wherein the photoaging causes deep wrinkles, yellow and leathery surface, hardening of the skin, elastosis, roughness, dyspigmentations, blotchy skin, or a combination thereof.

18. The method of claim 13, wherein the administering is accomplished by topical application.

19. A method for the prevention of skin aging comprising administering to a mammal a therapeutically effective amount of an LXR modulator.

20. The method of claim 19, wherein the administering is accomplished by topical application.

21. A method of counteracting UV photodamage comprising contacting a skin cell exposed to UV light with a therapeutically effective amount of an LXR modulator.

22. The method of claim 21, wherein the skin cell is a keratinocyte or a fibroblast.

23. A method of identifying an LXR modulator capable of inducing an anti-skin aging effect comprising:
(a) providing a sample containing LXR;
(b) contacting the sample with a test compound; and
(c) determining whether the test compound induces TIMP1 expression, induces ASAH1 expression, induces SPTLC1 expression, induces SMPD1 expression, induces LASS2 expression, induces TXNRD1 expression, induces GPX3 expression, induces GSR expression, induces CAT expression, induces ABCA1 expression, induces ABCA2 expression, induces ABCA12 expression, induces ABCA13 expression, induces ABCG1 expression, induces decorin expression, inhibits TNFα expression, inhibits MMP1 expression, inhibits MMP3 expression, inhibits IL-8 expression, or a combination thereof through an LXR-based mechanism.
